(19) United States
(12) Patent Application Publication
(10) Pub. No.: US 2024/0384043 A1
(43) Pub. Date: Nov. 21, 2024

(54) METHODS OF PRODUCING CROSSLINKED HYALURONIC ACID HYDROGELS WITH DESIRABLE GEL PROPERTIES

(71) Applicant: GALDERMA HOLDING SA, Zug (CH)

(72) Inventors: Åke ÖHRLUND, Uppsala (SE); Morgan KARLSSON, Almunge (SE)

(73) Assignee: GALDERMA HOLDING SA, Zug (CH)

(21) Appl. No.: 18/790,315

(22) Filed: Jul. 31, 2024

Related U.S. Application Data
(63) Continuation of application No. PCT/IB2023/050852, filed on Jan. 31, 2023.
(60) Provisional application No. 63/305,513, filed on Feb. 1, 2022.

(51) Int. Cl. C08J 3/075; C08J 3/24

(52) U.S. Cl. CPC C08J 3/075; C08J 3/24; C08J 2305/08

(57) ABSTRACT

Described are glycosaminoglycan (GAG) hydrogels with desirable gel properties and methods of producing GAGs with desirable gel properties, particularly hyaluronic acid hydrogels from non-animal origin hyaluronic acid. Further described are esthetic compositions such as hydrogels containing crosslinked polysaccharides, and the use of such hydrogels in medical and/or cosmetic applications. The disclosure further is concerned with hyaluronic acid hydrogels produced from non-animal sourced hyaluronic acid to mitigate unwanted immune reactions

### RELATED APPLICATIONS

This application is a continuation of International PCT Application PCT/IB2023/050852 filed Jan. 31, 2023, which application claims priority under 35 U.S.C. § 119 (c) to U.S. Provisional Application No. 63/305,513 filed Feb. 1, 2022, the entire contents of which are incorporated herein by reference.

### FIELD

The present disclosure relates to the field of glycosaminoglycan (GAG) hydrogels and methods of producing GAGs with desirable gel properties, particularly hyaluronic acid hydrogels from non-animal origin hyaluronic acid. The present disclosure is further drawn to esthetic compositions such as hydrogels containing crosslinked polysaccharides, and the use of such hydrogels in medical and/or cosmetic applications such as implants for subcutaneous or intradermal injection, which may be used in humans in applications of esthetic dermatology and reparative or plastic surgery. More specifically, the present disclosure is concerned with hyaluronic acid hydrogels produced from non-animal sourced hyaluronic acid to mitigate unwanted immune reactions.

### BACKGROUND

Hydrogels are widely used in medicine-prepared by chemical crosslinking polymers to form large polymeric networks. While both monomeric and minimally polymerized polysaccharides both absorb water to the point of saturation, polysaccharides dissolve at the point of saturation while hydrogels comprising the same polysaccharides, albeit crosslinked, can typically absorb water without dissolving, resulting in a swelling of the hydrogel.

All glycosaminoglycans (GAGs) are negatively charged long linear heteropolysaccharides that have a capacity to absorb large amounts of water. Hyaluronic acid, chondroitin, and chondroitin sulfate are well-known biocompatible GAGs utilized in medical and cosmetic applications. One of the most widely used biocompatible polymers for medical use is hyaluronic acid, and derivatives thereof. Modifying hyaluronic acid molecules through crosslinking and other means is necessary to modulate various desirable properties to produce a hydrogel that that tailored to a specific application, treatment, duration, etc.

Producing hydrogels GAGs, such as hyaluronic acid results in a suitable filler for multiple types of medical or cosmetic applications; however, there is not one catchall hydrogel that is sufficient to serve all of the various medical and aesthetic applications of hydrogels. Methods of modulating various desirable aspects of the hydrogel allows for the creating of hydrogels for a variety of different purposes. The aim of the present disclosure is to fulfill a need for producing hydrogels with different desirable properties such as hydrogels with high or low elasticity (elastic modulus-G′), high or low firmness, etc.

### SUMMARY OF THE DISCLOSURE

The present disclosure is generally drawn to methods of creating hydrogels with desirable properties for particular medical/esthetic applications, wherein the desirable properties include increased or decreased elasticity and increased or decreased firmness. The present disclosure is further drawn to hydrogel compositions produced by the methods.

In some aspects, the disclosure is drawn to a process for preparing a product comprising crosslinked glycosaminoglycan (GAG) molecules, the process comprising: (a) crosslinking GAG molecules with a diepoxide crosslinking agent under alkaline conditions at a temperature of less than 20° C., and (b) swelling the crosslinked GAG molecules in an aqueous solution. In some aspects, the disclosure is drawn to a process for preparing a product comprising crosslinked glycosaminoglycan (GAG) molecules, the process comprising: (a) crosslinking GAG molecules with a diepoxide crosslinking agent under alkaline conditions at a temperature of greater than 45° C. for at least 2 hours, and (b) swelling the crosslinked GAG molecules in an aqueous solution.

In some aspects, the diepoxide crosslinking agent is 1,4-butanediol diglycidyl ether. In some aspects, the crosslinking in (a) occurs for at least about 4 hours. In some aspects, the crosslinking in (a) occurs for at least about 2 hours. In some aspects, the crosslinking in (a) occurs for at least 1 hour, at least 2 hours, at least 4 hours, at least 6 hours, at least 8 hours, at least 12 hours, at least 16 hour, at least 20 hours, at least 24 hours, at least two days, at least 3 days, at least 4 days, at least 5 days, at least 6 days, at least 7 days, at least 8 days, at least 9 days, at least 10 days, at least 12 days, at least 15 days, at least 20 days, or any range or value therein. In some aspects, the swelling occurs for about 20 hours. In some aspects, the swelling occurs for at least 10 hours. In some aspects, the swelling occurs until the gel is at a concentration of about 20 mg/g of the aqueous solution. In some aspects, gel particles are formed from the crosslinked GAG molecules prior to (b). In some aspects, the gel particles are formed from the crosslinked GAG molecules after (b). In some aspects, the crosslinking occurs at about 5° C. In some aspects, the crosslinking occurs at about 50° C.

In some aspects, the alkaline conditions are a pH of at least 8.5. In some aspects, the alkaline conditions are a pH of at least 9. In some aspects, the alkaline conditions are the presence of sodium hydroxide. In some aspects, the sodium hydroxide concentration during crosslinking is about 0.5 w/w % about 10 w/w %. In some aspects, the alkaline conditions are neutralized after crosslinking.

In some aspects, the GAG molecules are hyaluronic acid (HA) molecules. In some aspects, the HA concentration during crosslinking is about 5 w/w % to about 50 w/w % (e.g., about 25 w/w %). In some aspects, the aqueous solution comprises a dissolved salt. In some aspects, the dissolved salt is NaCl. In some aspects, the aqueous solution is a buffered solution. In some aspects, the aqueous solution comprises a phosphate buffer.

In some aspects, during cross-linking, the GAG chains (e.g., HA chains) exhibit a degradation rate of about 500× lesser than the degradation rate of GAG chains (e.g., HA chains) during cross-linking at a temperature of about 50° C. In some aspects, the product is a gel that exhibits increased firmness as compared to a product that was crosslinked at a temperature of about 50° C. In some aspects, the product is a gel that exhibits longer GAG chains as compared to a product that was crosslinked at a temperature of about 50° C. In some aspects, the product exhibits an elastic modulus about 50× greater than the elastic modulus of a product that was crosslinked at a temperature of about 50° C.

In some aspects, the disclosure is drawn to a process for preparing a product comprising crosslinked hyaluronic acid (HA) molecules, the process comprising: (a) crosslinking HA molecules with 4-butanediol diglycidyl ether crosslinking agent under alkaline conditions at a temperature of about 5° C. or about 50° C. for between about 2 to about 200 hours, (b) dividing the crosslinked HA molecules into particles of less than about 1 cm (e.g., equal or less than about 9 mm, equal to or less than about 8 mm, equal to or less than about 7 mm, equal to or less than about 6 mm, equal to or less than about 5 mm, equal or less than about 4 mm, equal or less than about 3 mm, equal or less than about 2 mm, equal or less than about 1 mm, equal or less than about 800 μm, equal to or less than about 700 μm, equal to or less than about 600 μm, equal to or less than about 500 μm, equal to or less than about 400 μm, equal to or less than about 300 μm, equal to or less than about 200 μm, equal to or less than about 100 μm, equal to or less than about 90 μm, equal to or less than about 80 μm, equal to or less than about 70 μm, equal to or less than about 60 μm, equal to or less than about 50 μm, equal to or less than about 40 μm, equal to or less than about 30 μm, equal to or less than about 20 μm, equal to or less than about 10 μm, equal to or less than about 1 μm, equal to or less than about 900 nm, equal to or less than about 800 nm, equal to or less than about 700 nm, equal to or less than about 600 nm, or equal to or less than about 500 nm, or smaller), (c) swelling the crosslinked HA from (b) in a solution comprising NaCl (e.g., about 0.9% NaCl) until the particles reach a concentration of about 20 mg/g, and (d) collecting and sterilizing the crosslinked HA from (c).

In some aspects, the disclosure is drawn to a crosslinked HA product produced by any one of the processes described herein. In some aspects, the disclosure is drawn to a composition comprising any one of the crosslinked HA products described herein. In some aspects, the disclosure is drawn to a crosslinked GAG product produced by any one of the processes described herein. In some aspects, the crosslinked product is crosslinked via ether bonds to the diepoxide crosslinking agent. In some aspects, the crosslinked product is crosslinked via ester bonds to the diepoxide crosslinking agent.

In some aspects, the crosslinked product is a dermal filler product. In some aspects, the crosslinked GAG product further comprises one or more pharmaceutical agents. In some aspects, the one or more pharmaceutical agents is selected from one or more of a local anesthetic, an anti-inflammatory drug, a hormone, and an antibiotic drug. In some aspects, the composition comprising any one of the crosslinked GAG products described herein further comprises living cells.

In some aspects, the disclosure is drawn to a method of administering any one of the compositions or products described herein to a mammal in need thereof. In some aspects, the mammal is a human.

In some aspects, the disclosure is generally drawn to a method of cosmetically treating skin, which comprises administering to the skin a hydrogel product according to any one of the hydrogel compositions or products described herein.

In some embodiments, the crosslinking occurs at about 5° C. or at about 50° C.

The following detailed description is exemplary and explanatory, and is intended to provide further explanation of the invention.

### BRIEF DESCRIPTION OF THE FIGURES

FIG. 1 depicts the HA (left), MoD (middle), and product CrR concentration (right) for the product resulting from the NASHA crosslinking process at three different temperatures and crosslinking times.

FIG. 2 depicts the HA chain length of the crosslinked product, as Mw from reference experiments (not autoclaved). The three data points correspond to the NASHA crosslinking process at three different temperatures and crosslinking times.

FIG. 3 depicts the gel product parameters estimating obtained gel strength; G′ (left), SwF (middle), and GelC (right). The three data points for each box correspond to the NASHA crosslinking process at three different temperatures and crosslinking times.

FIG. 4 depicts the degradation rate as 1/Mw against time for the 5° C., 23° C., and 50° C. crosslinking temperatures.

FIG. 5 depicts the Arrhenius plot of HA degradation rates at the 5° C., 23° C., and 50° C. crosslinking temperatures.

FIG. 6 depicts the extrapolation to 500 h of 1/Mw vs time (left) and Mw vs time (right) for the 5° C., 23° C., and 50° C. crosslinking temperatures.

FIG. 7 depicts the difference (left) and the ratio (right) of Mw over time for the 5° C., 23° C., and 50° C. crosslinking temperatures.

### DETAILED DESCRIPTION OF THE DISCLOSURE

### I. Definitions

While the following terms are believed to be well understood by one of ordinary skill in the art, the following definitions are set forth to facilitate explanation of the presently disclosed subject matter.

The term “a” or “an” may refer to one or more of that entity, i.e., can refer to plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

Reference throughout this specification to “one embodiment”, “an embodiment”, “one aspect”, or “an aspect” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

As used herein, the terms “about” or “approximately” when preceding a numerical value indicates the value plus or minus a range of 10% of the value.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

As used herein, a “control” is an alternative sample used in an experiment for comparison purpose. A control can be “positive” or “negative.” A “control sample” or “reference sample” as used herein, refers to a sample or reference that acts as a control for comparison to an experimental sample. For example, an experimental sample comprises compound A, B, and C in a vial, and the control may be the same type of sample treated identically to the experimental sample, but lacking one or more of compounds A, B, or C.

As used herein, the term “effective amount” refers to a quantity sufficient to achieve a desired therapeutic and/or prophylactic effect, e.g., an amount which results in the prevention of one or more outcomes, or an increase in one more outcomes.

As used herein, the terms “individual”, “patient”, or “subject” can be an individual organism, a vertebrate, a mammal, or a human. In a preferred aspect, the individual, patient, or subject is a human.

As used herein, the term “hydrogel” refers to a water-containing three dimensional hydrophilic polymer network or gel in which the water is the continuous phase and in which the water content is greater than 50% (w/w).

As used herein, the phrase “biocompatible polymer” is a polymer having degradation products that are compatible with living tissue, or that may have beneficial biological properties. The biocompatible polymer may be biocompatible in itself, and/or may be synergistically biocompatible when employed in conjunction with a biologically active agent.

As used herein, the term “hyaluronic acid polymer” refers to a polymer comprising repeat disaccharide subunits of hyaluronan, where the repeat units may be derivatized at one or more positions of the D-glucuronic acid and/or the D-N-acetylglucosamine unit of the disaccharide repeat subunit. A hyaluronic acid polymer is meant to encompass hyaluronic acid (also referred to as hyaluronan), derivatized hyaluronic acid, salts forms, hyaluronic acid linker complexes, and hyaluronic acid conjugates.

As used herein, the phrase, “hyaluronic acid” is meant to refer to unmodified or non-derivatized hyaluronic acid.

As used herein, the phrase “hyaluronic acid derivative” or “derivatized hyaluronic acid” or “modified hyaluronic acid” refers to hyaluronic acid that has been derivatized by reaction with, e.g., one or more small chemical moieties such as divinyl sulfone or the like.

As used herein, the phrase “lightly crosslinked” or “having a low degree of crosslinking” means that the crosslinking reaction occurs such that about 30% to about 70% of the available crosslinking sites are reacted to generate the final crosslinked gels, where the modified hyaluronic acid starting material used to form the gel possesses 10% or less of its hydroxyl groups in activated/derivatized form, to thereby provide an hydrogel that is considered overall to be lightly crosslinked.

As used herein, the phrase “particle size” or “bead size” refer to diameters, and are typically determined by sieve analysis. The sizes or ranges described typically correspond to a sieve or mesh opening size. One may refer to a particle size conversion chart to determine the size, e.g., in mm, corresponding to a particular mesh or screen number.

As used herein, the phrase “soft tissue” refers to tissues that connect, support, or surround other structures and organs of the body. Soft tissue includes muscles, fibrous tissues, and fat.

As used herein, the phrase “soft tissue augmentation” refers to any type of volume augmentation of soft tissues, including, but not limited to facial contouring (e.g., more pronounced checks, chin, or lips), correction of concave deformities (e.g., post-traumatic or HIV-associated lipoatrophy), and correction of deep age-related facial folds. Thus, soft tissue augmentation may be used for cosmetic purposes or for medical purposes, such as those following trauma or degenerative disease. Soft tissue augmentation further refers to dermal filling, body contouring, and gingival filling.

As used herein, the term “drug,” or “pharmaceutically active agent” or “bioactive agent” or “active agent” as used interchangeably, means any organic or inorganic compound or substance having bioactivity and adapted or used for therapeutic purposes. Proteins, hormones, anti-cancer agents, analgesics, anesthetics, small molecule chemical compounds and mimetics, oligonucleotides, DNA, RNA and gene therapies are included under the broader definition of “drug”. As used herein, reference to a drug, as well as reference to other chemical compounds herein, is meant to include the compound in any of its pharmaceutically acceptable forms, including isomers such as diastereomers and enantiomers, salts, solvates, and polymorphs, particular crystalline forms, as well as racemic mixtures and pure isomers of the compounds described herein, where applicable.

As used herein, the phrase “non-animal origin” refers to a source that excludes animals, but includes sources such as yeast, bacteria, or synthetic.

As used herein, “NASHA” is (1) an acronym for non-animal stabilized hyaluronic acid, (2) a particular type of hyaluronic acid hydrogel, and (3) the process of producing the particular type of hyaluronic acid hydrogel. A NASHA hydrogel is produced by crosslinking hyaluronic acid—not from an animal—with a small amount of 1,4-butanediol diglycidyl ether (BDDE), preferably, as the crosslinker such that the final concentration of hyaluronic acid is in the range of about 10 to about 30 mg/ml and the degree of modification with the crosslinker is less than 1.0 mol %, preferably about 1 mol %.

As used herein, the term “microbead” is used interchangeably with microdrop, microdroplet, microparticle, microsphere, nanobead, nanodrop, nanodroplet, nanoparticle, nanosphere, particle, etc. A typical microbead is approximately spherical and has an number average cross-section or diameter in the range of between 1 nanometer to 1 millimeter. Though, usually the microbeads of the one embodiment will be made with a desired size in a much more narrow range, e.g., they will be fairly uniform. The microbeads preferably have a diameter in the range of about 100-1,000 nanometer; or in the range of 1,000 nanometer to 1,000 micrometer. The size-distribution of the microbeads will be low and the polydispersibility narrow.

As used herein, the term “bioresorbable” refers to a degradation event or events-bioresorbable substances may dissolve, may be phagocytized, or may simply degrade over a period of time such that the substances are cleared from the body, organ, tissue, location, or cell over a period of time. The substances or degradation products thereof may be metabolized, incorporated into other molecules or compounds, or excreted.

As used herein, the term “aseptic” refers to something that is free or freed from pathogenic microorganisms.

As used herein, the term “sterile” refers to something that is free of living organisms, generally free of living microorganisms.

As used herein, the term “injectable” refers to the ability to inject a composition of the present disclosure through a needle.

As used herein, the terms “MW” or “Mw” refer to the mass average molecular mass.

As used herein, the term “MWapp” refers to apparent MW, which is a simulated value for the molecular weight of GAGs in hydrogels.

As used herein, the term “SwF” refers to the swelling factor analysis in saline, which is the volume of saline for a 1 gram gel that has swelled to its maximum-usually represented in mL/g).

As used herein, “gel content” or “GelC” refer to the percentage in the proportion of the total HA that is bound in gel form-further described as the amount of HA in a sample that does not pass through a 0.22 micrometer filter. The GelC is calculated from the amount of HA that is collected in the filtrate and is reported as the percentage of the total amount of HA in the gel sample.

As used herein, “SwD” refers to the swelling degree, which is the inverted concentration of gel-form GAG in a gel that is fully swollen in 0.9% saline, e.g., the volume or mass of a fully swollen gel that can be formed per gram of dry crosslinked GAG. The SwD generally describes the maximal liquid-absorbing (0.9% saline) capability of the product. SwD is preferably expressed in g/g, mL/g, or as a dimensionless number.

$$SwD = \frac{mass ( fully swollen gel )}{mass ( gel - form GAG in fully swollen gel )}$$

The SwD may also be expressed as

$$SwD = \frac{[ GAG ] * GelC}{SwF}$$

As used herein, “CrR” refers to the effective crosslinking ratio that was analyzed with LC-SEC-MS, more specifically defined as

$$CrR = \frac{mol crosslinked crosslinker with amide bonds}{mol linked crosslinker with amide bonds}$$

A CrR of 1.0 indicates that all of the crosslinker has crosslinked.

As used herein, “Cₘᵢₙ” is the minimum theoretical GAG concentration—the concentration of gel-form GAGs in a gel that is fully swollen in 0.9% saline, typically expressed in mg/g or mg/mL.

$$The {{C}_{min}}^{- 1} = SwD$$

As used herein, “C_{final}” is the intended concentration of the GAG in the final hydrogel product. In some aspects, C_{final} is greater than 2×Cₘᵢₙ.

The present technology is not to be limited in terms of the particular aspects described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

### II. Hydrogels and Methods of Making Hydrogels

Fillers such as dermal fillers have been used to repair, restore or augment hard or soft tissue contour defects of the body due to aging, injury, or acquired or congenital deformities of the face, body and internal organs. Fillers may be natural or synthetic substances that are used to reduce wrinkles and/or fine lines, restore lost volume, hydrate the skin, soften nasolabial folds, augment and contour lips, improve scars (depressed, hypertrophic and keloid scars), strengthen weakened vocal cords, and provide other soft tissue improvements. Substances that have been utilized include fat, paraffin, human collagen, bovine collagen, silicone, hyaluronic acids, lactic acids, and glycolic acids. In 1981, a new era in soft tissue fillers emerged with the FDA approval of bovine collagen. Since then, many soft tissue fillers have emerged. The dramatic increase in the number of current and investigational fillers has been fueled by many factors including improvements in biotechnology and an emphasis on cosmetic appearance in society. With the introduction of newer fillers, there has been an ongoing need to evaluate their risk/benefit profiles and define their limitations in order to maximize patient cosmetic outcomes and safety. Common filler/hydrogel compositions include GAGs such as hyaluronic acid.

Methods of producing GAG hydrogels are disclosed in PCT publication numbers WO2017/114867, WO2017/114861, WO2017/114864, WO2017/114865, WO2014/206500, WO2017/016917, WO2019/121688, and WO1997/004012; US Patent Publication Numbers US2020/0316260, US2019/0023812A1, US2019/0016830A1, US2019/0023855A1, US2021/0155763, and US2007/0066816A1; and U.S. Pat. Nos. 8,858,999, 6,831,172, 8,887,243, 6,703,444, 10,077,321, and 5,827,937. In some aspects, the methods of the present disclosure may be applied to any of the GAG crosslinking processes described herein.

### Hydrogel Production Method 1

In some aspects, a NASHA hydrogel is produced by crosslinking hyaluronic acid—not from an animal—with a small amount of 1,4-butanediol diglycidyl ether (BDDE), preferably, as the crosslinker such that the final concentration of hyaluronic acid is in the range of about 10 to about 30 mg/ml and the degree of modification with the crosslinker is less than 1.0 mol %, preferably about 1 mol %.

In some aspects, the NASHA hydrogel is produced by activation of the polymer (HA) under alkaline conditions. 10 g of hyaluronic acid prepared from a bacterial or yeast culture are dissolved in 100 ml of 1% NaOH, pH>9. The crosslinking agent in the form of BDDE is added to a concentration of 0.2%. The solution is incubated at 40° C. for about 4 hours. In some aspects, the NASHA hydrogel can be produced under acidic conditions at a pH of about 2 to about 6 due to the addition of about 1% acetic acid to the solution instead of NaOH.

The hydroxide or acidic incubates are diluted to a volume which was about twice the volume finally desired, to about 0.5 to about 1% and are neutralized.

It has been shown that by changing the temperature during NASHA crosslinking, while adjusting time to obtain equal level of crosslinking, the resulting HA chain length of the crosslinked gel can be controlled, resulting in widely different gel properties. Crosslinking using NASHA conditions at temperatures 5° C., 23° C. and 50° C. showed that 5° C. resulted in a very firm gel with long HA chains, while crosslinking at 50° C. resulted in a very soft gel with short HA chains. The 23° C. crosslinking expectedly resulted in typical NASHA gel properties.

Since all three gels were equal in level of crosslinking and HA concentration, the difference in gel properties were considered mainly due to HA chain length. The final HA gel chain length for the 5° C. crosslinked gel was about two times that of the 50° C. crosslinked gel. The resulting G′ differed by a factor of 50, demonstrating that differences in chain length of the final HA gel can have a great impact on gel properties.

The degradation rates differed by 500× between the 5° C. and the 50° C. experiments, yielding an Arrhenius slope of 104 KJ/mol. The difficulty to envision the effect of degradation over time, due to the non-linearity of the molecular weight of the HA (Mw) over time, is discussed and demonstrated herein.

It has been observed that the sensitivity to change in temperature (activation energy, Ea, Arrhenius slope) is higher for the degradation (depolymerization) of HA than for the reaction rate of BDDE. This led to the idea that by changing the crosslinking temperature, while adjusting reaction time to allow the same level of crosslinking, the degradation level of HA during crosslinking can be adjusted. The expected result is that crosslinking at lower temperatures would result in a gel with longer HA chains, and conversely, that crosslinking at higher temperatures would result in a gel with shorter HA chains. In the instance of at least BDDE as the crosslinker, the degradation rate of HA is much more temperature sensitive than the BDDE crosslinking rate. When utilizing low temperatures with HA crosslinking, the high molecular weight of the HA can be retained, thus allowing for the the use of smaller amounts of the crosslinker while maintaining the strength of the HA gel. When utilizing high temperatures with HA crosslinking, the molecular weight of the HA can be decreased, making the gel softer, even if a higher degree of crosslinking is desired. Even temperatures below 0° C. are capable of being used with the methods described herein.

Conventional wisdom in the art is that at high pH, significant degradation of the HA backbone is expected during crosslinking. The conventional approach in the art is to accept this degradation, since high pH is required to achieve crosslinking. Methods according to the present disclosure advantageously utilize low-temperature processing to reduce degradation of the backbone during cross-linking.

To demonstrate this, three crosslinking experiments were performed at temperatures 5° C., 23° C. and 50° C., where the reaction time was adjusted to give the same level of crosslinking. The crosslinking conditions, apart from time and temperature, were set according to NASHA. The length of the HA chains in the crosslinked gels were estimated by analysing the Mw of HA solutions from reference “crosslinking” experiments with no crosslinker. The properties of the final crosslinked gel were studied by testing the parameters rheology, swelling factor (SwF) and gel content (GelC). The level of crosslinking was studied by measuring the degree of modification (MoD).

In some aspects, the crosslinker is selected from the group consisting of divinyl sulfone, multiepoxides, and diepoxides. In some aspects, the crosslinker is a diepoxide selected from 1,4-butanediol diglycidyl ether (BDDE), 1,2-ethanediol diglycidyl ether (EDDE), or diepoxyoctane.

A common route for crosslinking hyaluronic acid is using a diglycidyl ether, e.g., BDDE. As an alternative, amide coupling using a di- or multiamine functional crosslinker together with a coupling agent is an attractive route for preparing crosslinked hyaluronic acid molecules useful for hydrogel products.

In some aspects, the crosslinker itself contributes to maintained or increased properties of the hydrogel, for example when crosslinking with a structure that correlates to hyaluronic acid or when crosslinking with a structure with high water retention properties.

### Hydrogel Production Method 2

In some aspects, there is provided an injectable gel product comprising a first, inner phase of a plurality of cross-linked glycosaminoglycan (GAG) gel particles embedded in a second, outer phase of a cross-linked glycosaminoglycan (GAG) gel; wherein the second, outer phase is in the form of particles; wherein the Degree of Modification (MoD) of the gel of the first, inner phase is 0.15 or lower, and wherein the MoD of the gel of the second, outer phase is lower than the MoD of the gel of the first, inner phase, and wherein the MoD is the molar amount of bound cross-linking agent(s) relative to the total molar amount of repeating GAG disaccharide units.

An injectable gel product refers to the gel being capable to flow and be injected through a syringe, such as a through a syringe having a needle of fine diameter as generally required in cosmetic surgery. The needle may be of the type 27 gauge. Thus, the injectable gel product may be ready to be packaged in a syringe.

The injectable gel product may be regarded as biphasic, e.g., comprising a first, inner phase comprising the plurality of gel particles embedded in a second, outer gel phase. The inner phase may have a higher concentration of GAGs, may be more firm and/or have a higher degree of cross-linking.

The injectable gel product may also be cohesive or exhibit cohesivity, e.g., as defined in WO 11086458 A1.

A glycosaminoglycan (GAG) is a negatively charged heteropolysaccharide chain which have a capacity to absorb large amounts of water. The GAG may for example be sulfated or non-sulfated glycosaminoglycans such as hyaluronan, chondroitin sulphate, heparan sulphate, heparosan, heparin, dermatan sulphate and keratan sulphate. In some embodiments the GAG is hyaluronic acid, chondroitin or chondroitin sulfate.

The gel of the inner phase and/or the outer phase may be a hydrogel. That is, they may be regarded as a water-insoluble, but substantially dilute, cross-linked systems of GAG molecules when subjected to a liquid, typically an aqueous liquid.

The gel of the first inner phase and the gel of the second outer phase are cross-linked. The gels thus comprise a continuous shaped network of GAG molecules which is held together by the covalent cross-links, physical entangling of the GAG chains and various interactions, such as hydrogen bonding, van der Waals forces and electrostatic interactions.

As an example, the degree of cross-linking of the gel product may be such that the charging ratio is less than 0.1, preferably less than or equal to 0.02, when preparing the gels of both the first, inner phase and the second, outer phase.

The gel of the first inner phase and the gel of the second outer phase are further in the form of particles, e.g., in the form of fragments that may have any type of shape, regular or irregular. The sizes may be non-uniform, e.g., the gel product may comprise particles of different sizes. Furthermore, the gel particles of the outer phase comprise a plurality of inner gel particles. In other words, the particle size of the outer gel is larger than the particle size of the inner gel.

It is to be understood that the first, inner phase of the plurality of cross-linked glycosaminoglycan (GAG) gel particles themselves may comprise further phases, such as smaller particles embedded within the first, inner phase of the plurality of cross-linked glycosaminoglycan (GAG) gel particles, and such smaller particles may have even smaller particles embedded within, and so on.

In some aspects, the Degree of Modification (MoD) of the gel of the first, inner phase is 0.10 or lower, such as 0.05 or lower, such as 0.02 or lower. In some aspects, the Degree of Modification (MoD) of the gel of the second, outer phase is 0.10 or lower. Gels with such a softness may be suitable for contact with tissue after injection.

However, the MoD of the second outer phase may be above 0.05, such as above 0.05, but less than the MoD of the gel of the inner phase. As an example, the MoD of the gel of the second, outer phase may be between 0.08 and 0.095. Gels having such a MoD have been found to be firm enough to hold together as a cohesive gel but still be perceived as soft upon injection.

A relevant physical property of a cross-linked GAG gel product is the volume of liquid that the gel can absorb and is related to the structural stability of the gel, often referred to as gel strength or firmness. Traditional expressions for the liquid absorption are swelling, swelling capacity, liquid retention capacity, swelling degree, swelling factor, maximum liquid uptake and maximum swelling. Throughout this text, the term swelling factor (SwF) will be used.

When the gel is subjected to non-precipitating conditions, it is possible to determine its swelling factor, or inversely its minimum concentration (Cmin), i.e., the GAG concentration when the gel product is fully swollen. Further addition of liquid will not dilute the gel further, e.g., the gel cannot be indefinitely diluted like a solution of free molecules. Firmer (low-swelling) gels are generally expected to have a longer half-life in vivo than softer (high-swelling) gels.

In some aspects, the Swelling factor (SwF) of the gel of the second, outer phase is above 3.0, such as above 4.0. Such a gel may be perceived as soft for the tissue.

However, the swelling factor of the gel of the second, outer phase may still be below 10.0, e.g., between 3.0 and 10.0, such as between 3.0 and 5.0. A gel having such a swelling factor may be soft enough to be perceived as soft, but be firm enough to retain a plurality of the inner gel particles embedded in the gel during manufacture, such as after Particle Size Reduction (PSR) of the outer gel. PSR is a process in which a gel is pushed through a grid, thereby breaking the gel into particles having an average particles size corresponding to the through holes of the grid.

In some aspects, the gels of the first, inner phase and the second, outer phase are cross-linked to each other. Thus, the gels may be cross-linked to each other so as to form a single, cohesive gel, in contrast to having the inner gel particles dispersed in the outer gel. The cross-links between the inner and outer gels may be performed while crosslinking the outer gel. This may be advantageous in that it may prevent inner gel particles from separating from the outer gel before the outer gel has been fully dissolved after injection.

In some aspects, the Degree of Modification (MoD) is substantially homogenous throughout the second, outer phase and substantially homogenous throughout the first, inner phase. Thus, the degree of cross-linking may be substantially homogeneous in both gel phases, such as constant when seen in a direction from the center of the gel and outwards. This is thus in contrast to a gel in which the degree of crosslinking varies within the gel.

The outer gel particles may comprise at least five inner gel particles, such as at least ten inner gel particles, such as at least twenty inner gel particles. In some aspects, the outer gel particle size is at least three times the size of an inner particle size. Furthermore, the inner particles may have an average size that is less than 0.200 mm.

In some aspects, the dry weight content of the cross-linked glycosaminoglycan (GAG) of the first, inner phase is at least 25% of the total dry weight content of glycosaminoglycans (GAGs) in the inner and outer phase, such as at least 50% of the total dry weight content of glycosaminoglycans (GAGs) in the inner and outer phase. Such gels may be soft enough to provide an initial softness in vivo but have high enough inner particle content to provide a second function once the outer gel has dissolved. As an example, the dry weight content of the cross-linked glycosaminoglycan (GAG) of the first, inner phase may be at least 60% of the total dry weight content of glycosaminoglycans (GAGs) in the inner and outer phase. As a further example, the dry weight content of the cross-linked glycosaminoglycan (GAG) of the first, inner phase may be between 65% and 95% of the total dry weight content of glycosaminoglycans (GAGs) in the inner and outer phase. In some aspects, the glycosaminoglycan (GAG) is hyaluronic acid.

Unless otherwise provided, the term “hyaluronic acid” encompasses all variants and combinations of variants of hyaluronic acid, or hyaluronan, of various chain lengths and charge states, as well as with various chemical modifications That is, the term also encompasses the various hyaluronate salts of hyaluronic acid, such as sodium hyaluronate. Various modifications of the hyaluronic acid are also encompassed by the term, such as oxidation, e.g., oxidation of CH2OH groups to COOH; periodate oxidation of vicinal hydroxyl groups, optionally followed by reduction or imine formation etc; reduction, e.g., reduction of COOH to CH2OH; sulphation; deamidation, optionally followed by deamination or amide formation with new acids; esterification; substitutions with various compounds, e.g., using a cross-linking agent or a carbodiimide; including coupling of different molecules, such as proteins, peptides and active drug components, to hyaluronic acid; and deacetylation.

As an example, the hyaluronic acid may be a chemically unmodified hyaluronic acid or hyaluronate salt, preferably sodium hyaluronate, having an average molecular weight in the range of 0.1-10 MDa, preferably 0.8-5 MDa, more preferably 0.8-1.2 MDa. It is preferred that the hyaluronic acid is obtained from non-animal origin, preferably bacteria.

In some aspects, the cross-linking of the inner and/or the outer gel is performed using one or more polyfunctional cross-linking agent(s) individually selected from the group consisting of divinyl sulfone, multiepoxides and diepoxides. In certain embodiments, the polyfunctional cross-linking agent(s) is individually selected from the group consisting of 1,4-butanediol diglycidyl ether (BDDE), 1,2-ethanediol diglycidyl ether (EDDE) and diepoxyoctane. In specific embodiments, the polyfunctional cross-linking agent is 1,4-butanediol diglycidyl ether (BDDE). Thus, in some aspects, the crosslinking is ether bonds.

In some aspects, crosslinking of the glycosaminoglycan is achieved by amide coupling of glycosaminoglycan molecules. The amide coupling may be performed using a di- or multinucleophile functional crosslinker. Amide coupling using a di- or multiamine functional crosslinker together with a coupling agent is an attractive route for preparing crosslinked glycosaminoglycan molecules useful in the present invention. Crosslinking can be achieved using a non-carbohydrate based di- or multinucleofile crosslinker, for example hexamethylenediamine (HMDA), together with a glycosaminoglycan. Thus, the crosslinker comprising at least two nucleophilic functional groups may for example be a non-carbohydrate based di- or multinucleophilic crosslinker or a carbohydrate based di- or multinucleophilic crosslinker.

A preferred group of di- or multinucleophile functional crosslinker includes homo- or heterobifunctional primary amines, hydrazines, hydrazides, carbazates, semi-carbazides, thiosemicarbazides, thiocarbazates and aminoxy groups.

Carbohydrate based di- or multinucleophilic crosslinkers may be advantageous, since they provide a hydrogel product based entirely on carbohydrate type structures or derivatives thereof, which minimizes the disturbance of the crosslinking on the native properties of the glycosaminoglycans. The crosslinker itself can also contribute to maintained or increased properties of the hydrogel, for example when crosslinking with a structure that correlates to hyaluronic acid or when crosslinking with a structure with high water retention properties.

In some aspects, the di- or multinucleofile crosslinker is an at least partially deacetylated polysaccharide, e.g., an acetylated polysaccharide which has been at least partially deacetylated to provide a polysaccharide having free amine groups. An at least partially deacetylated glycosaminoglycan, can be crosslinked either alone or in combination with a second glycosaminoglycan, whereby the deacetylated glycosaminoglycan itself acts as the di- or multinucleofile crosslinker.

In some aspects, the glycosaminoglycan molecules of the first, inner phase and/or the glycosaminoglycan molecules of the second outer phase are covalently crosslinked via crosslinks comprising a spacer group selected from the group consisting i) the spacer group and ii) the binding groups formed upon reaction of the functional groups of the crosslinker with the carboxylic acid groups on the GAG.

In some embodiments, crosslinking via crosslinkers provides a hydrogel product based entirely on carbohydrate type structures or derivatives thereof, which minimizes the disturbance of the crosslinking on the native properties of the glycosaminoglycans. The crosslinker is preferably well defined in terms of structure and molecular weight. In some embodiments, the spacer is mono-disperse or has a narrow molecular weight distribution. Using well defined crosslinkers together with a highly efficient condensation reaction allows the product to be assembled in a controlled fashion. The crosslinker itself can also contribute to maintained or increased properties of the hydrogel, for example when crosslinking with a structure that correlates to hyaluronic acid or when crosslinking with a structure with high water retention properties.

In some aspects, at least 90% of the bonds between glycosaminoglycan molecules and crosslinks are amide bonds. Furthermore, less than 5% of the bonds between glycosaminoglycan molecules and crosslinks may be ester bonds. The first and/or second glycosaminoglycan may further be crosslinked using a peptide coupling reagent. Crosslinking using a peptide coupling agent is advantageous over many other common crosslinking methods (e.g., BDDE crosslinking) since it allows for crosslinking to be performed at neutral pH with minimal degradation of the glycosaminoglycan molecules.

In some aspects, the peptide coupling reagent is selected from the group consisting of triazine-based coupling reagents, carbodiimide coupling reagents, imidazolium-derived coupling reagents, Oxyma and COMU.

According to some embodiments, the peptide coupling reagent is a triazine-based coupling reagent. According to some embodiments, the triazine-based coupling reagent is selected from the group consisting of 4-(4,6-dimethoxy-1,3,5-triazin-2-yl)-4-methylmorpholinium chloride (DMTMM) and 2-chloro-4,6-dimethoxy-1,3,5-triazine (CDMT). According to some embodiments, the triazine-based coupling reagent is DMTMM. According to some aspects, the peptide coupling reagent is a carbodiimide coupling reagent. According to some embodiments, the carbodiimide coupling reagent is N-(3-dimethylaminopropyl)-N′-ethylcarbodiimide (EDC) combined with N-hydroxysuccinimide (NHS).

The injectable gel product may also comprise a portion of GAG molecules which are not crosslinked, e.g., not bound to a three-dimensional crosslinked GAG molecule network. However, it is preferred that at least 50% by weight, preferably at least 60% by weight, more preferably at least 70% by weight, and most preferably at least 80% by weight, of the GAG molecules injectable gel product form part of a crosslinked GAG molecule network.

As a second aspect of the invention, there is provided a method of producing an injectable gel product, comprising the steps of: a) cross-linking a first glycosaminoglycan (GAG) with a first crosslinking agent to produce a gel, wherein the charging ratio of crosslinking agent to disaccharide unit is below 0.15; b) preparing particles of the gel from step a); c) mixing the glycosaminoglycan (GAG) gel particles of step b) with a second glycosaminoglycan (GAG) to provide a mixture; d) cross-linking the mixture of step c) with a second crosslinking agent to obtain cross-linking between the glycosaminoglycans (GAGs) of the second, outer phase, thereby providing a gel having a first, inner phase of the cross-linked glycosaminoglycan (GAG) gel particles, embedded in a gel of the second, outer phase comprising the second glycosaminoglycan (GAG), and e) preparing injectable particles of the gel from step d), each such particle containing a plurality of the cross-linked GAG gel particles of the first, inner phase.

The method of the second aspect is advantageous in that it is a convenient process for producing a combination gel, e.g., for embedding a densely crosslinked, and therefore firm, material into a lightly crosslinked, and therefore soft, material. Such a combination gel may give stronger lifting capacity in the tissue. The inventors have found that having a charging ratio in step a) of below 0.15, e.g., when producing the “inner” GAG gel particles, and an even lower charging ratio in step d), e.g., when embedding the inner GAG gel particles in the outer matrix, facilitates the formation of a gel product that may give stronger lifting capacity in the tissue.

Further, the method of the second aspect of the invention is advantageous in that it allows for an effective process for producing the injectable gel-product, since low amounts of cross-linking agent may be used.

The steps of embedding already crosslinked particles within a gel that is subsequently crosslinked may be repeated for any number of times, such as at least two times or at least three times. Thus, step d) may further comprise preparing particles of the provided gel and these prepared particles may be used in a repeated step c) in which they are mixed with, e.g., a third glycosaminoglycan (GAG) to provide a mixture, that is subsequently crosslinked in a repeated step d) to form a gel. Particles of this gel may yet again be mixed with further GAGs, and so on. do not check

Thus, steps c) and d) may be repeated any number of times. The crosslinking agent used in the crosslinking steps may be as discussed in the first aspect of the invention above. As an example, the crosslinking in step a) and/or step b) may result in ether bonds. The crosslinking in step d) may be performed using the same crosslinking conditions as the crosslinking in step a). The charging ratio in step d) may be the same as in step a) or different. Further, the crosslinking in step d) may be performed using the same crosslinking agent as in step a) or using a different crosslinking agent than the crosslinking agent used in step a). Consequently, the first and second crosslinking agents may be the same or different-Further, the cross-linking of steps a) and/or d) may be performed by an initial addition of cross-linking agent to increase the homogeneity of crosslinkage in the first and/or outer gel. The charging ratio of step a) may be less than 0.10, such as less than 0.05.

As an example, the charging ratio of step a) may be between 0.002-0.10, such as between 0.005-0.10, such as between 0.005-0.05, such as between 0.005-0.03, such as between 0.01-0.3.

In some aspects, the charging ratio of crosslinking agent to disaccharide unit in step d) is less than 0.10, such as less than 0.05. The inventors have found that such a low charging ratio when producing the outer gel matrix may still provide a gel product with suitable degrading characteristics in situ. In some aspects, the charging ratio of crosslinking agent to disaccharide unit in step d) is less than the charging ratio of step a). In some aspects, the cross-linking of step d) further comprises obtaining cross-linking between the gels of the first, inner phase and the second, outer phase. Thus, the second crosslinking agent used in step d) may further aid in crosslinking the outer gel matrix to the embedded gel particles, and may thus aid in retaining the embedded gel particles within the gel when preparing the injectable particles in step c).

In some aspects, step c) comprises mixing the glycosaminoglycan (GAG) gel particles of step b) in dry state with the second glycosaminoglycan (GAG) in dry state. The gel particles in dry state may for example be in powder form or in the form of dry entangled strings. Mixing in dry state may be advantageous in that it provides for good mixing between the first and second GAGs, e.g., it provides for an even distribution of the gel particles of the first inner phase within the outer phase. However, step c) may comprise first dissolving the glycosaminoglycan (GAG) gel particles of step b) and mixing the solution with a solution comprising the second glycosaminoglycan (GAG). In some aspects, the step of preparing particles of step b) further comprises precipitating and drying said particles prepared of the gel of step a).

Precipitation of the inner gel particles is advantageous in that it further increases the possibilities of washing the gel particles of the first inner phase, e.g., to decrease the number of by-products, such as unreacted cross-linking agents. The precipitation in itself may act as a cleaning step; e.g., unwanted by-products may be separated from the gel particles during the actual precipitation. Further, precipitation of the inner gel particles may make the inner gel particles more accessible for crosslinking agents during the second step of crosslinking, e.g., during step d). This means that lower amounts of crosslinking agent may be used in the second crosslinking step, e.g., precipitation increases the effective use of the crosslinking agents and thus increases the effectiveness of the whole method. Moreover, a precipitated powder may be stored and therefore, the second crosslinking of step d) does not have to be performed immediately after the precipitation. Thus, step c) may be performed at least 1 hour after the precipitation, such as at least 5 hours, such as at least 10 hours, such as at least 24 hours, such as at least 48 hours after precipitation. Hence, a step of precipitating and drying the particles prepared of the gel of step a) increases the flexibility of the process.

Consequently, a step of precipitation is advantageous in that it allows for cleaning of the gel particles, the process becomes more flexible in that the two cross-linking steps may be divided in more distinct and separate crosslinking steps and the overall effectiveness of the process in increased due to the use of lower amounts of crosslinking agent.

In some aspects, the mixture of step c) contains at least 45% by dry weight of the GAG gel particles obtained in step b), such as at least 50% by dry weight of the GAG gel particles obtained in step b). Thus, the dry weight content of the cross-linked glycosaminoglycan (GAG) of the first, inner phase may be at least 50% of the total dry weight content of glycosaminoglycans (GAGs) in step c). As an example, the mixture of step c) may contain at least 60% by dry weight of the GAG gel particles obtained in step b). As a further example, the mixture of step c) may contain between 65% and 95% by dry weight of the GAG gel particles obtained in step b). Thus, the dry weight content of the cross-linked glycosaminoglycan (GAG) of the first, inner phase may be between 65% and 95% of the total dry weight content of glycosaminoglycans (GAGs) in the inner and outer phase.

As a third aspect of the invention, there is provided an aqueous composition comprising an injectable gel product according to any embodiment of the first aspect above and optionally a buffering agent.

The aqueous composition may be suitable for injection. The aqueous composition may typically contain a physiological salt buffer. The composition may further comprise other suitable additives, such as local anaesthetics (e.g., lidocaine hydrochloride), anti-inflammatory drugs, antibiotics, and other suitable supportive medications, e.g., bone growth factors or cells.

Administration of the injectable gel product or the aqueous composition may be performed in any suitable way, such as via injection from standard cannulae and needles of appropriate sizes or surgical insertion, e.g., in the case of administration of a film. The administration is performed where the soft tissue augmentation is desired, such as the chin, checks or elsewhere in the face or body.

Thus, as a fourth aspect of the invention there is provided a pre-filled syringe, which is pre-filled with a sterilized, injectable gel product according to any embodiment of the first aspect above or a sterilized aqueous composition according to the third aspect above.

The gel product may be kept in precipitated form in the syringe be brought to its non-precipitated form prior to injection or in the body following injection thereof.

The gel product may further be autoclavable, since this is the most convenient way of sterilising the final product. This allows for preparation of a sterilized, injectable gel product.

In some aspects, the injectable gel product or the aqueous composition according to the first and third aspects above are useful as a medicament or medical device in a medical or surgical method. According to a further aspect, there is provided the use of an injectable gel product according to the first aspect above or the aqueous composition according to third aspect above in cosmetic or medical surgery. Put another way, there is provided an injectable gel product or an aqueous composition for use in cosmetic or medical surgery.

In some aspects, the use is in cosmetic surgery selected from dermal filling and body contouring. In some other embodiments, the use is as a medicament in the treatment of, and/or in medical surgery selected from, dermal filling, body contouring, prevention of tissue adhesion, formation of channels, incontinence treatment, and orthopaedic applications.

In some aspects, there is provided a method of treatment of a subject undergoing cosmetic or medical surgery, involving administration of an injectable gel product according to the first aspect above or the aqueous composition according to third aspect above to a subject in need thereof.

In some aspects, the subject is undergoing cosmetic surgery selected from dermal filling and body contouring. In certain other embodiments, the subject is undergoing medical surgery, or medical treatment, for a condition selected from dermal filling, body contouring, prevention of tissue adhesion, formation of channels, incontinence treatment, and orthopaedic applications.

The glycosaminoglycan may itself act as a crosslinker in a crosslinked gel, e.g., when a deacetylated glycosaminoglycan is used. In such cases, the Degree of Modification (MoD), which is a relevant property when using crosslinking agents other than the glycosaminoglycan itself, may correspond to the degree of deacetylation of the glycosaminoglycan. Thus, all embodiments and examples of MoD discussed in relation to the previous aspects above may also be relevant when the glycosaminoglycan itself acts as a crosslinker, but then as degree of deacetylation of the glycosaminoglycan.

Thus, as a general aspect of the present invention, there is provided an injectable gel product comprising a first, inner phase of a plurality of cross-linked glycosaminoglycan (GAG) gel particles embedded in a second, outer phase of a cross-linked glycosaminoglycan (GAG) gel; wherein the second, outer phase is in the form of particles; wherein maximum amount of crosslinkages relative to the total molar amount of repeating GAG disaccharide units of the gel of the first, inner phase is 0.15 or lower, and wherein the maximum amount of crosslinkages relative to the total molar amount of repeating GAG disaccharide units of the gel of the second, outer phase is lower than the maximum amount of crosslinkages relative to the total molar amount of repeating GAG disaccharide units of the gel of the first, inner phase.

The maximum amount of crosslinkages relative to the total molar amount of repeating GAG disaccharide units may for example be the MoD if a separate crosslinking agent other than the GAG itself is used for crosslinking or the degree of deacetylation if the GAG itself acts as the crosslinker. Furthermore, when using the glycosaminoglycan itself acts as a crosslinker in the crosslinked gel, the alternative aspects discussed below may become relevant. The terms and definitions used in relation to these alternative aspects are as discussed in relation to the previous aspects above.

Thus, as an alternative aspect of the present invention, there is provided an injectable gel product comprising a first, inner phase of a plurality of cross-linked glycosaminoglycan (GAG) gel particles embedded in a second, outer phase of a cross-linked glycosaminoglycan (GAG) gel; wherein the second, outer phase is in the form of particles and wherein the glycosaminoglycan (GAG) of the first inner phase and/or the glycosaminoglycan (GAG) of the second outer phase comprises crosslinks in which the glycosaminoglycan itself acts as a di- or multinucleofile crosslinker.

In some aspects, the degree of deacetylation of the gel of the first, inner phase is 0.15 or lower, and wherein the degree of deacetylation of the gel of the second, outer phase is lower than the degree of deacetylation of the gel of the first, inner phase.

In some aspects, only the glycosaminoglycan (GAG) of the first inner phase comprises crosslinks in which the glycosaminoglycan itself acts as a di- or multinucleofile crosslinker. In some aspects, only the glycosaminoglycan (GAG) of the second outer phase comprises crosslinks in which the glycosaminoglycan itself acts as a di- or multinucleofile crosslinker. In some aspects, the glycosaminoglycan (GAG) of the first inner phase and the glycosaminoglycan (GAG) of the second outer phase comprises crosslinks in which the glycosaminoglycan itself acts as a di- or multinucleofile crosslinker. As an example, a deacetylated glycosaminoglycan itself may act as the di- or multinucleofile crosslinker.

Thus, the glycosaminoglycan itself acting as a di- or multinucleophilic crosslinker may be an at least partially deacetylated glycosaminoglycan, e.g., an acetylated glycosaminoglycan which has been at least partially deacetylated to provide a glycosaminoglycan having free amine groups. An at least partially deacetylated glycosaminoglycan can be crosslinked either alone or in combination with a second glycosaminoglycan, whereby the deacetylated glycosaminoglycan itself acts as the di- or multinucleophilic crosslinker.

As a further alternative aspect of the present invention, there is provided a method for producing an injectable gel product, comprising the steps of: a) cross-linking a first glycosaminoglycan (GAG) to produce a gel, b) preparing particles of the gel from step a); c) mixing the glycosaminoglycan (GAG) gel particles of step b) with a second glycosaminoglycan (GAG) to provide a mixture; d) cross-linking the mixture of step c) to obtain cross-linking between the glycosaminoglycans (GAGs) of the second, outer phase, thereby providing a gel having a first, inner phase of the cross-linked glycosaminoglycan (GAG) gel particles, embedded in a gel of the second, outer phase comprising the second glycosaminoglycan (GAG), and e) preparing injectable particles of the gel from step d), each such particle containing a plurality of the cross-linked GAG gel particles of the first, inner phase.

The crosslinking of step a) and/or step d) may be achieved using an at least partially deacetylated glycosaminoglycan, either alone or in combination with a second glycosaminoglycan, whereby the deacetylated glycosaminoglycan itself acts as the di- or multinucleofile crosslinker.

The degree of deacetylation of the first glycosaminoglycan in step a) may be below 0.15. The degree of deacetylation of the second glycosaminoglycan in step d) may be less than the degree of deacetylation of the first glycosaminoglycan.

By the term “at least partially deacetylated” as used herein regarding the glycosaminoglycan, it is meant a glycosaminoglycan comprising N-acetyl groups in which at least some of the N-acetyl groups have been cleaved off, resulting in the formation of free amine groups in the glycosaminoglycan. By “at least partially deacetylated” as used herein, we mean that a significant portion of the N-acetyl groups of the glycosaminoglycan, particularly at least 1%, preferably at least 2%, at least 3%, at least 4%, at least 5%, of the N-acetyl groups of the glycosaminoglycan have been converted to free amine groups. More preferably, at least 3% of the N-acetyl groups of the glycosaminoglycan have been converted to free amine groups.

In some aspects, the at least partially deacetylated glycosaminoglycan may have a degree of acetylation of less than 99%, preferably less than 98%, less than 97%, less than 97%, less than 96%, less than 95%, less than 94% or less than 93%. The crosslinking of deacetylated glycosaminoglycan may be performed with the aid of a coupling agent, such as a peptide coupling reagent. In some aspects, the peptide coupling reagent is selected from the group consisting of triazine-based coupling reagents, carbodiimide coupling reagents, imidazolium-derived coupling reagents, Oxyma and COMU. According to some embodiments, the peptide coupling reagent is a triazine-based coupling reagent. According to some embodiments, the triazine-based coupling reagent is selected from the group consisting of 4-(4,6-dimethoxy-1,3,5-triazin-2-yl)-4-methylmorpholinium chloride (DMTMM) and 2-chloro-4,6-dimethoxy-1,3,5-triazine (CDMT). According to some embodiments, the triazine-based coupling reagent is DMTMM. According to some embodiments, the peptide coupling reagent is a carbodiimide coupling reagent. According to some embodiments, the carbodiimide coupling reagent is N-(3-dimethylaminopropyl)-N′-ethylcarbodiimide (EDC) combined with N-hydroxysuccinimide (NHS).

Consequently, step a) may comprise the sub steps: i) providing a solution comprising an at least partially deacetylated glycosaminoglycan and optionally a further glycosaminoglycan; ii) activating carboxyl groups on the at least partially deacetylated glycosaminoglycan and/or the optional further glycosaminoglycan with a coupling agent, to form activated glycosaminoglycans; iii) crosslinking the activated glycosaminoglycans via their activated carboxyl groups using amino groups of the at least partially deacetylated glycosaminoglycans to provide a gel of glycosaminoglycans crosslinked by amide bonds.

As a further example, the second glycosaminoglycan may be at least partially deacetylated glycosaminoglycan and step d) may comprise the sub steps i) activating carboxyl groups on the at least partially deacetylated second glycosaminoglycan with a coupling agent, to form activated glycosaminoglycans; ii) crosslinking the activated glycosaminoglycans via their activated carboxyl groups using amino groups of the at least partially deacetylated glycosaminoglycans to provide the second, outer phase comprising the second glycosaminoglycan (GAG) crosslinked by amide bonds.

In some aspects, the crosslinked GAG is step a) and/or step d) is obtained by: 1) crosslinking at least partially deacetylated GAG to partially deacetylated GAG in the presence of a coupling agent using free amine and carboxylic acid groups present in the at least partially deacetylated GAGs; or 2) crosslinking at least partially deacetylated GAG to a non-deacetylated GAG in the presence of a coupling agent using free amine groups present in the at least partially deacetylated GAG and carboxylic acid groups present in the GAG.

In some aspects, the at least partially deacetylated glycosaminoglycan has a degree of acetylation of 99% or less, preferably 98% or less, preferably 97% or less, preferably 96% or less, preferably 95% or less, preferably 94% or less, preferably 93% or less, and a weight average molecular weight of 0.1 MDa or more, preferably 0.5 MDa or more. According to some embodiments, the at least partially deacetylated glycosaminoglycan is obtained by a method for at least partial deacetylation of a glycosaminoglycan, comprising: a1) providing a glycosaminoglycan comprising acetyl groups; a2) allowing the glycosaminoglycan comprising acetyl groups to react with hydroxylamine (NH2OH) or a salt thereof at a temperature of 100° C. or less for 2-200 hours to form an at least partially deacetylated glycosaminoglycan; and a3) recovering the at least partially deacetylated glycosaminoglycan.

In some aspects, the hydrogels are created by intermingling different hydrogels. In some aspects, the different hydrogels are different in that they comprise a different degree of crosslinking, comprise a different swelling factor, and/or comprise a different G′ (Pa) (the measure of elastic modulus). In some aspects, the different hydrogels, once intermingled, are subject further crosslinking. In some aspects, the different hydrogels, prior to intermingling, are formed into various sizes of particles. In some aspects, this results in both differences in (1) particle size and (2) crosslinking degree, elastic modulus, and/or swelling factor. In some aspects, intermingling hydrogels may include blending different, separately-prepared hydrogels. In some aspects, intermingling hydrogels may include cross-linking separately-prepared hydrogels.

In some aspects, a hydrogel composition comprises at least 2 different hydrogels. In some aspects, a hydrogel composition comprises at least 3 different hydrogels. In some aspects, a hydrogel composition comprises at least 4 different hydrogels. In some aspects, a hydrogel composition comprises at least 5 different hydrogels. In some aspects, a hydrogel composition comprises at least 6 different hydrogels. In some aspects, each of the hydrogels differ by (1) particle size and/or (2) crosslinking degree, elastic modulus, and/or swelling factor.

In some aspects, a hydrogel composition comprises a first hydrogel having a first crosslinking degree, a second hydrogel having a second crosslinking degree, and/or a third hydrogel having a third crosslinking degree; wherein the first, second, and/or third hydrogels are intermingled and formed into particles of the same or different size.

### Hydrogel Production Method 3

In some aspects of the present disclosure, a product that is manufactured by the method is a cross-linked HA. The method provides ether cross-links between the HA chains, which creates a continuous shaped network of HA molecules which is held together by the covalent cross-links, physical entangling of the HA chains and various interactions, such as hydrogen bonding, van der Waals forces and electrostatic interactions. The cross-linked HA product according to the invention is a gel, or a hydrogel. That is, it can be regarded as a water-insoluble, but substantially dilute, cross-linked system of HA molecules when subjected to a liquid, typically an aqueous liquid. The resulting cross-linked HA product is preferably biocompatible. This implies that no, or only very mild, immune response occurs in the treated individual.

Since cross-linked HA gel products are highly complex chemical structures, they are typically characterised by a combination of their chemical structures and their physical properties. The deviation in chemical structure from unmodified HA is typically reported as degree of modification, modification degree, cross-linking degree, cross-linking index or chemical modification, which all relate to the amount of cross-linking agent covalently bound to the HA. Throughout this text, the term degree of modification will be used.

In some aspects, the most relevant physical properties of the cross-linked HA gel product are the volume of liquid that the gel can absorb and the rheological properties of the gel. Both properties describe the structural stability of the gel, often referred to as gel strength or firmness, but while the absorption of liquid can be determined for a dry gel, the rheological properties have to be measured on a gel that is swollen to a desired concentration. The measurement yields the resistance of the gel to deformation in terms of elastic modulus (G′) and viscous modulus (G″). A high gel strength will give a large resistance to deformation of the gel product swollen to a desired concentration. According to a first aspect, the present invention provides a process for manufacturing a cross-linked HA gel product. The process is comprising at least (a) a step of preparing an aqueous mixture of the reactants; and (b) a subsequent step of allowing the reactants to cross-link and form a cross-linked HA gel product in a single reaction.

In the initial step (a) of preparing an aqueous mixture of the reactants, an aqueous mixture of HA and a cross-linking agent is prepared. According to the present invention, the HA should be present in higher concentrations than what is commonly used. The dissolved HA constitutes more than 10% (w/w) of the final mixture. The dissolved HA preferably constitutes 15% or more, such as 20% or more, or 25% or more (w/w) of the final mixture. The dissolved HA preferably constitutes 50% or less, such as 45% or less, such as 40% or less, or even 35% or less (w/w) of the final mixture. Preferred ranges of dissolved HA are, e.g., 10-50%, such as 15-45%, such as 20-40%, such as 25-35% (w/w) of the final mixture.

The HA is dissolved in the aqueous solution. By the terms “dissolved” and “solution” is understood that the HA is present in a homogeneous mixture with a liquid, in which mixture energetically favourable interactions occur. Addition of liquid to the solution lowers the concentration of the dissolved HA substrate. The solution is aqueous, e.g., it contains water to a major extent except for the HA and the cross-linking agent. The aqueous solution may simply comprise, or even consist, of the HA substrate dissolved in water containing inorganic ions and the cross-linking agent.

Further in the initial step (a) of preparing an aqueous mixture of the reactants, the mixture is prepared by dissolving the HA in an aqueous solution containing a relatively high concentration of hydroxide, preferably 1-10% (w/w). This is typically achieved by dissolving inorganic hydroxide, such as hydroxides of alkali metals, e.g., NaOH, KOH and LiOH; and hydroxides of alkaline earth metals, e.g., Mg(OH)₂ and Ca(OH)₂, in water prior to adding the cross-linking agent and the HA. It is not critical which inorganic hydroxide is used, and hydroxides of alkali metals, e.g., NaOH, KOH, are preferred, e.g., for reasons of economy. A preferred inorganic hydroxide is NaOH. The resulting aqueous solution is preferably comprising more than 1%, such as 1.5% or more, such as 2% or more, such as more than 2.00%, such as 2.1% or more, such as 2.5% or more (w/w) inorganic hydroxide. Importantly, the resulting aqueous solution is at the same time comprising 10% or less, such as 8% or less, such as 6% or less, such as 5% or less, such as 4% or less (w/w) inorganic hydroxide. Preferred ranges of inorganic hydroxide in the solution are, e.g., 1.5-8%, such as 1.5-6% (w/w); such as 2-4% (w/w) inorganic hydroxide, especially more than 2.00%, such as 2.1% or more (w/w) inorganic hydroxide which provides strong and firm HA gel products.

The hydroxide concentration in the mixture can also be considered as corresponding to the hydroxide concentration resulting from the above-mentioned concentrations of NaOH, e.g., an hydroxide concentration corresponding to more than 1%, such as 1.5% or more, such as 2% or more, such as more than 2.00%, such as 2.1% or more, such as 2.5% or more (w/w) NaOH, and at the same time corresponding to 10% or less, such as 8% or less, such as 6% or less, such as 5% or less, such as 4% or less (w/w) NaOH. Preferred ranges of hydroxide concentration in the solution are corresponding to, e.g., 1.5-8%, such as 1.5-6% (w/w); such as 2-4% (w/w) NaOH, especially, more than 2.00%, such as 2.1% or more NaOH.

It is particularly preferred to combine a high concentration of HA and gradually higher concentrations of inorganic hydroxide within the interval presented as surprisingly useful. It has been experimentally observed that stronger gels can be obtained using more than 2.00%, such as 2.1% or more, such as 2.5% or more or even 4% or more (w/w) inorganic hydroxide in combination with a dissolved HA concentration of 20% or more, or 25% or more (w/w) of the final mixture.

The cross-linking agent employed in the present invention is selected from multiepoxides and diepoxides, preferably diepoxides. Preferred diepoxide cross-linking agents according to the invention include 1,4-butanediol diglycidyl ether (BDDE), 1,2-ethanediol diglycidyl ether (EDDE) and diepoxyoctane, preferably BDDE. Under the strongly basic conditions employed in the cross-linking-step, these cross-linking agents provide ether cross-links between the HA chains. It is not critical which amount of cross-linking agent is used, as long as the cross-linking agent is not completely consumed in the subsequent cross-linking reaction.

The cross-linking agent can be added to the aqueous solution prior to, at the same time as, or after addition of the HA. The cross-linking agent and/or the HA can also be to the aqueous solution added in portions in any suitable order.

In the subsequent step (b) of allowing the reactants to cross-link and form a cross-linked HA gel product, the aqueous mixture prepared in step (a) is subjected to cross-linking conditions, which typically involves a desirable time and temperature. In general terms, this cross-linking step can be made using any high concentration of HA and suitable concentration of cross-linking agent as set out hereinabove, and the time and temperature can vary. The dissolved HA is allowed to react with the cross-linking agent for a suitable time to obtain a cross-linked HA gel product. For avoidance of doubt, this implies that the subsequent step (b) is the only, i.e., single cross-linking step.

The cross-linking step is typically carried out at a temperature of 10-75° C., e.g., 10-40° C., such as 10-35° C. or 10-30° C. but it is preferred that the step is carried out at 15-35° C., such as 15-30° C., and especially at room temperature, e.g., 20-25° C. Preferred temperature ranges are 10-50° C., such as 18-40° C. The reaction time is suitably in the range of 2-40 h, such as 4-36 h. A longer reaction time than 2 h is useful for reproducibility, especially at larger scale. Longer reaction time than 40 h yields a gel with lower gel strength or may even disrupt the gel entirely. The reaction time is preferably in the range of 8-30 h, such as 12-24 h, e.g., 16-24 h. In a preferred embodiment, the cross-linking step is performed at 15-35° C. for 2-40 h, such as at room temperature for 16-24 h, to obtain a cross-linked HA gel with excellent implantation properties, in particular gel strength.

Due to the cross-linking, the resulting HA product is a continuous network of interconnected and entangled HA chains which absorbs liquid (swells) and forms a gel. That is, it can be regarded as a water-insoluble, but substantially dilute cross-linked system of HA molecules when subjected to a liquid, typically an aqueous liquid. The gel is mostly liquid by weight and can contain, e.g., 90-99.9% water, but it behaves like a solid due to a three-dimensional cross-linked HA network within the liquid. Due to its significant liquid content, the shaped gel is structurally flexible and similar to natural tissue, which makes it very useful as a scaffold in tissue engineering and for tissue augmentation.

The swelling of the resulting HA gel can be allowed to proceed until the gel is fully swollen and no further liquid can be absorbed, or it can be interrupted at an earlier stage to obtain a partially swollen gel. A partially swollen gel can be useful as an intermediate for further processing of the gel, for instance further mechanical production of gel structures of a desired size and shape can be performed. It may also be convenient to use a partially swollen shaped gel product during implantation thereof at a desired site to facilitate administration and minimize patient discomfort and to enhance the lifting capacity by use of the remaining swelling capacity.

If desired, other substances, such as local anaesthetics (e.g., lidocaine hydrochloride), anti-inflammatory drugs, antibiotics and other suitable supportive medications, e.g., bone growth factors or cells, may be added after the cross-linked HA product has been obtained. In some aspects, the method may also comprise one or more further steps. Optionally, the manufacturing process involves a step of isolating the cross-linked HA product, e.g., by filtration, dialysis or precipitation in a precipitating medium to remove cross-linking agent which has not been incorporated into the HA gel product.

It is particularly preferred to include a step of precipitating the HA gel product after the cross-linking step has been terminated to wash away residual (soluble) cross-linking agent which has not been incorporated into the product. The precipitated HA gel product is then dissolved in any suitable aqueous buffer. Using a higher cross-linker concentration is useful to provide a strong gel, and the increased residual amount of (soluble) cross-linking agent is readily removed with a precipitation step. As a result, a firm and pure HA gel is obtained with excellent implantation properties. Without being limited thereto, useful precipitation media include pentane, hexane, cyclohexane, 1,4-dioxane, N,N-dimethylformamide, N,N-dimethylacetamide, ethyl acetate, acetamide, diethyl ether, tetrahydrofurane, acetonitrile, methyl ethyl ketone, acetone, lower alkyl alcohols, e.g., methanol, ethanol, propanol, isopropanol and butanol. A preferred group of precipitation media is the lower alkyl alcohols. The term lower alkyl alcohol includes primary, secondary and tertiary alkyl alcohols having from one to six carbon atoms, e.g., C₁₋₆ alkyl alcohols. Specific examples of lower alkyl alcohols include methanol, ethanol, denatured spirit, n-propanol, isopropanol, n-butanol, isobutanol, and t-butanol. Preferred lower alkyl alcohols are methanol and ethanol, in particular ethanol, due to price, availability and easy handling.

Optionally, the manufacturing process involves a further step of sterilizing the cross-linked HA product, e.g., by autoclaving, radiation, heating etc., so as to obtain a sterile cross-linked HA product.

According to a related aspect, the present invention provides a cross-linked HA gel product obtainable, or even obtained, by the process according to the invention. The product can advantageously be further characterized by one or more of the following features.

The amount of attached cross-linking agent(s) can be quantified by and reported as the degree of modification (MoD), i.e., the molar amount of bound cross-linking agent(s) relative to the total number of repeating HA disaccharide units. It is preferred that the cross-linked hyaluronic acid product according to the invention has a degree of modification of 1-90 cross-linking agent units per 1000 disaccharide units (0.1-9%), preferably 1-40 cross-linking agent units per 1000 disaccharide units (0.1-4%).

### Hydrogel Production Method 4

In some aspects, the product that is manufactured by the method is a shaped cross-linked hyaluronic acid. The method provides cross-links between the hyaluronic acid chains when they have been arranged in a desirable shape, which creates a continuous shaped network of hyaluronic acid molecules which is held together by the covalent cross-links, physical entangling of the hyaluronic acid chains and various interactions, such as hydrogen bonding, van der Waals forces and electrostatic interactions. The shaped cross-linked hyaluronic acid product according to the invention is a gel, or a hydrogel. That is, it can be regarded as a water-insoluble, but substantially dilute, cross-linked system of hyaluronic acid molecules when subjected to a liquid, typically an aqueous liquid.

The resulting shaped cross-linked hyaluronic acid product is preferably biocompatible. This implies that no, or only very mild, immune response occurs in the treated individual. In the Examples, there is provided a method of determining the biocompatibility of a hyaluronic acid product, and results from testing the biocompatibility of a cross-linked hyaluronic acid product according to the invention in rats.

In some aspects, the method comprises at least three steps: a preparation step, a precipitation step, and a cross-linking step. In certain embodiments, the method is consisting of these three steps.

In the first method step, a hyaluronic acid substrate is provided. As set out above, the term “hyaluronic acid substrate” encompasses all variants and combinations of variants of hyaluronic acid, or hyaluronan, of various chain lengths and charge states, as well as with various chemical modifications. It is preferable that the hyaluronic acid substrate is a chemically unmodified hyaluronic acid or hyaluronate salt, preferably sodium hyaluronate, having an average molecular weight in the range of 0.5-10 MDa, preferably 0.8-5 MDa, more preferably 1.5-3 MDa or 2-3 MDa. It is preferred that the hyaluronic acid is obtained from non-animal origin, preferably bacteria.

The hyaluronic acid substrate is dissolved in a first liquid medium, which is an aqueous solution. By the terms “dissolved” and “solution” is understood that the hyaluronic acid substrate is present in a homogeneous mixture with a liquid, in which mixture energetically favorable interactions occur. Addition of liquid to the solution lowers the concentration of the dissolved hyaluronic acid substrate. The solution is aqueous, i.e., it contains water. The aqueous solution may simply consist of the hyaluronic acid substrate dissolved in water. It is preferable that the aqueous solution contains 40-100 vol % water and 0-60 vol % of lower alkyl alcohol(s). The term “lower alkyl alcohol” includes primary, secondary and tertiary alkyl alcohols having from one to six carbon atoms, e.g., C₁₋₆ alkyl alcohols. Specific examples of lower alkyl alcohols include methanol, ethanol, denatured spirit, n-propanol, isopropanol, n-butanol, isobutanol, and t-butanol. Preferred lower alkyl alcohols are methanol and ethanol, in particular ethanol, due to price, availability and easy handling. The lower alkyl alcohol concentration is preferably in the range of 0-40%, such as 0-20%, 10-30% or 20-40% ethanol, with corresponding adjustments to the water component. The pH of this aqueous solution is suitably 6 or higher, such as 9 or higher.

Optionally, the first method step further involves arranging the hyaluronic acid substrate in a desired shape, such as a particle, a fibre, a string, a strand, a net, a film, a disc and a bead, which optionally is hollow or contain different layers of material. This may be accomplished in various ways, e.g., molding and extrusion. Extrusion of the hyaluronic acid substrate typically involves pressing the hyaluronic acid substrate solution through an opening of desired size. The extruded hyaluronic acid substrate spontaneously forms a precipitated fibre, string or strand. The dimensions, e.g., the thickness, of the fibre, string or strand can be controlled by varying the dimension or type of opening, e.g., using various opening diameters in the range of 0.1-2 mm or 14-30 G, the extrusion pressure, the extrusion speed and/or the hyaluronic acid concentration. By using other types of orifices and chinks, different shapes or structures can be produced. For instance, the hyaluronic acid can be precipitated as a film, a net, discs or beads.

In a preferred embodiment, the hyaluronic acid substrate solution is arranged in the desired shape on a hydrophobic surface, and the subsequent precipitation of the shaped hyaluronic acid substrate occurs on said hydrophobic surface. This is advantageous to avoid clogging of the shapes structures and to maintain the desired shape until it is fixed by the subsequent cross-linking step. Suitable hydrophobic surfaces are well known to the skilled person and include, e.g., fluorocarbons, polypropylene (PP), polyethylene terephthalate glycol-modified (PETG), polyethylene (PE), and polytetrafluoroethylene (PTFE).

This shape can be maintained throughout the manufacturing method and in the final product. It is preferred that the shape has an extension of less than 5 mm, preferably less than 1 mm, such as less than 0.5 mm or even less than 0.2 mm, in at least one dimension when the hyaluronic acid substrate is in precipitated form. This facilitates access for the cross-linking agent(s) to a high number of available binding sites of the precipitated hyaluronic acid products in the subsequent cross-linking step. It is also preferred that the shape is longitudinally extended and has a ratio between its longitudinal extension and its largest lateral extension of 5:1 or higher, such as 10:1 or higher, e.g., 20:1 or higher, and optionally 100,000:1 or lower, such as 25,000:1 or lower, e.g., 100:1 or lower. Since the longitudinally extended shape is maintained throughout the method and in the resulting product, the cross-linked product can be designed to avoid or decrease migration/displacement in vivo, but remains readily injectable. A suitable example of such shape is a fibre and the ratio between its length and its width is 5:1 or higher, such as 10:1 or higher, e.g., 20:1 or higher, and optionally 100,000:1 or lower, such as 25,000:1 or lower, e.g., 100:1 or lower. A preferred composition is comprising cross-linked strand/fibre-shaped hyaluronic acid products according to the invention, wherein more than 50% of the products have a ratio between its longitudinal extension and its largest lateral extension of 5:1 or higher, such as 10:1 or higher, e.g., 20:1 or higher, and optionally 100,000:1 or lower, such as 25,000:1 or lower, e.g., 100:1 or lower.

By way of example, a cross-linked hyaluronic acid product according to the invention with a single strand or fibre shape filling up a 20 ml syringe may have a thickness of 1 mm and a length of 25 m in swelled state, e.g., a ratio between its longitudinal extension and its largest lateral extension of 25,000:1. An example of a preferred composition comprises cross-linked strand/fibre-shaped hyaluronic acid products according to the invention, wherein more than 50% of the products have a longitudinal extension of more than 2 mm and a largest lateral extension of less than 0.2 mm, e.g., a ratio of 10:1 or higher.

The first method step is carried out without cross-linking, and this may be achieved by omitting cross-linking agents in this step and/or providing conditions that are not suitable for cross-linking. It is important to ensure that cross-linking does not occur until the preferred shape has been attained. This is advantageous for obtaining and maintaining a desired shape of the final product, since the shaping of the substrate is not limited by pre-existing cross-links, and all cross-links produced in the third step are directed to maintaining the desired shape of the product. It is preferred that the first step occurs in the absence of a cross-linking agent. This provides a good control of that the cross-linking does not occur in a dissolved phase and/or between method steps. It also ensures that the amount of cross-linking agent is tightly controlled and that the resulting products are homogenous in quality, since no cross-linking agent is reacted or lost in previous steps. Avoiding cross-linking, and in particular the addition of cross-linking agent in this step is useful to obtain a manufacturing process that is suitable for scaling up to an industrial scale and for providing products with homogenous quality.

In the second method step, the hyaluronic acid substrate is precipitated due to reduction of the solubility of the hyaluronic acid substrate. This is achieved by subjecting the hyaluronic acid substrate to a second liquid medium in which it is insoluble. The second liquid medium comprises an amount of one or more first water-soluble organic solvent(s) giving precipitating conditions for hyaluronic acid. The resulting solid precipitate falls out of the solute phase and can typically be separated from the remaining liquid by filtration, decanting, centrifugation, or manually using a pair of tweezers or the like. In one preferred embodiment, the precipitated hyaluronic acid substrate is also removed from the medium and dried. The precipitate can also be maintained suspended in the second liquid medium. Thus, in another preferred embodiment, the precipitated hyaluronic acid substrate is not subjected to drying. It is advantageous to achieve the precipitation of the hyaluronic acid substrate in a rapid fashion, e.g., by extruding or immersing the hyaluronic acid substrate in the second liquid medium in which it is insoluble.

The organic solvents that are used according to the invention are carbon-containing solvents and may exhibit a varying degree of polarity. Although termed “solvents”, it shall be understood that these organic solvents are utilized for balancing and shifting the solubility of hyaluronic acid during the manufacturing method. The hyaluronic acid may very well be dissolved in an organic solvent at a certain organic solvent concentration interval, but falls out and forms a precipitate when the organic solvent concentration is increased. For instance, the hyaluronic acid can be dissolved in a 50/50 (vol/vol) mixture of an organic solvent, e.g., a lower alkyl alcohol, and water, but falls out and forms a precipitate in a 90/10 (vol/vol) mixture. When subjected to non-precipitating conditions, e.g., a 50/50 or a 0/100 mixture, the hyaluronic acid returns to the non-precipitated, dissolved state. The skilled person is well aware that other factors may have an impact on the limiting organic solvent(s) concentration for precipitation of hyaluronic acid, such as temperature, pH, ion strength and type of organic solvent(s). The limiting concentration for precipitation of hyaluronic acid under given conditions is well known or can easily be determined by a skilled person in the field. By way of example, the limiting concentration for precipitation of hyaluronic acid (in mixture of water and ethanol) is approximately 70% ethanol.

Without being limited thereto, the organic solvents according to the invention can be selected from the group consisting of pentane, hexane, cyclohexane, 1,4-dioxane, N,N-dimethylformamide, N,N-dimethylacetamide, ethyl acetate, acetamide, diethyl ether, tetrahydrofurane, acetonitrile, methyl ethyl ketone, acetone, lower alkyl alcohols, e.g., methanol, ethanol, propanol, isopropanol and butanol, It is preferable that the organic solvents according to the invention are water-soluble. A preferred group of organic solvents is the lower alkyl alcohols. The term lower alkyl alcohol includes primary, secondary and tertiary alkyl alcohols having from one to six carbon atoms, e.g., C₁₋₆ alkyl alcohols. Specific examples of lower alkyl alcohols include methanol, ethanol, denatured spirit, n-propanol, isopropanol, n-butanol, isobutanol, and t-butanol. Preferred lower alkyl alcohols are methanol and ethanol, in particular ethanol, due to price, availability and easy handling.

It is suitable that the second liquid medium of the second method step is an aqueous medium, i.e., that it contains water to some extent. It is preferred that the second liquid medium contains 0-30 vol % water and 70-100 vol % of the first water-soluble organic solvent(s), preferably 0-10 vol % water and 90-100 vol % of the first water-soluble organic solvent(s). In certain embodiments, the concentration of the first water-soluble organic solvent(s) may be as high as 95%, such as 99% or even 99.5%, e.g., 99% methanol or ethanol. A high concentration of the first water-soluble organic solvent(s) is believed to be advantageous for achieving rapid precipitation. Thereby, an entangled structure can be achieved, which is likely to be advantageous for obtaining a gel product with desired properties.

The second method step is carried out without cross-linking, and this may be achieved by omitting cross-linking agents in this step and/or providing conditions that are not suitable for cross-linking. It is important to ensure that cross-linking does not occur until the preferred shape has been attained. This is advantageous for obtaining and maintaining a desired shape of the final product, since the shaping of the substrate is not limited by pre-existing cross-links, and all cross-links produced in the third step are directed to maintaining the desired shape of the product. It is preferred that the second step occurs in the absence of a cross-linking agent. This provides a good control of that the cross-linking does not occur in a dissolved phase and/or between method steps. It also ensures that the amount of cross-linking agent is tightly controlled and that the resulting products are homogenous in quality, since no cross-linking agent is reacted or lost in previous steps. Avoiding cross-linking, and in particular the addition of cross-linking agent, in this step is useful to obtain a manufacturing process that is suitable for scaling up to an industrial scale and for providing products with homogenous quality.

Optionally, the second method step further involves arranging the hyaluronic acid substrate in a desired shape, such as a particle, a fibre, a string, a strand, a net, a film, a disc and a bead, which optionally is hollow or contain different layers of material. This may be accomplished in various ways, e.g., molding and extrusion. This shape can be maintained throughout the manufacturing method and in the final product. It is preferred that the shape of the precipitated substrate has an extension of less than 5 mm, preferably less than 1 mm, such as less than 0.5 mm or even less than 0.2 mm, in at least one dimension when the hyaluronic acid substrate is in precipitated form. This facilitates access for the cross-linking agent(s) to a high number of available binding sites of the precipitated hyaluronic acid products in the subsequent cross-linking step. It is also preferred that the shape of the precipitated substrate is longitudinally extended and has a ratio between its longitudinal extension and its largest lateral extension of 5:1 or higher, such as 10:1 or higher, e.g., 20:1 or higher, and optionally 100,000:1 or lower, such as 25,000:1 or lower, e.g., 100:1 or lower. Since the longitudinally extended shape is maintained throughout the method and in the resulting product, the cross-linked product can be designed to avoid or decrease migration/displacement in vivo, but remains readily injectable. A suitable example of such shape is a fibre and the ratio between its length and its width is 5:1 or higher, such as 10:1 or higher, e.g., 20:1 or higher, and optionally 100,000:1 or lower, such as 25,000:1 or lower, e.g., 100:1 or lower. By way of example, a cross-linked hyaluronic acid product according to the invention with a single strand or fibre shape filling up a 20 mL syringe may have a thickness of 1 mm and a length of 25 m in swelled state, e.g., a ratio between its longitudinal extension and its largest lateral extension of 25,000:1.

The second method step may involve extrusion of the hyaluronic acid substrate into the second liquid medium, which comprises an amount of the first water-soluble organic solvent(s) giving precipitating conditions for hyaluronic acid. This is typically involving pressing the hyaluronic acid substrate solution through an opening of desired size into the second liquid medium. The extruded hyaluronic acid substrate spontaneously forms a precipitated fibre, string or strand in the second liquid medium. The dimensions, e.g., the thickness, of the fibre, string or strand can be controlled by varying the dimension or type of opening, e.g., using various opening diameters in the range of 0.1-2 mm or 14-30 G, the extrusion pressure, the extrusion speed and/or the hyaluronic acid concentration. By using other types of orifices and chinks, different shapes or structures can be produced. For instance, the hyaluronic acid can be precipitated as a film, a net, discs or beads. When a fibre, string or strand is formed, it is preferred that the ratio between its length and its average diameter is 5:1 or higher, such as 10:1 or higher, e.g., 20:1 or higher, and optionally 100,000:1 or lower, such as 25,000:1 or lower, e.g., 100:1 or lower. An advantage with the fibre/string/strand shape is that the fibers/strings/strands themselves can be entangled at the macroscopic level, causing a coil or ball effect which may be advantageous, e.g., for maintaining the integrity of an implant.

In the third method step, the precipitated hyaluronic acid substrate is for the first time subjected to cross-linking in a third liquid medium. The term “cross-linking” refers to introduction of stable covalent links (cross-links) between (at least) two different hyaluronic acid chains or (at least) two distinct sites of a single hyaluronic acid chain, which creates a continuous network of hyaluronic acid molecules. The cross-link may simply be a covalent bond between two atoms in the hyaluronic acid chains, e.g., an ether bond between two hydroxyl groups, or an ester bond between a hydroxyl group and a carboxyl group. The cross-link may also be a linker molecule that is covalently bound to two or more atoms of different hyaluronic acid chains or distinct sites of a single hyaluronic acid chain. Although cross-linking can occur spontaneously under certain conditions, the cross-linking typically involves use of a cross-linking agent, one or more, which facilitates and speeds up the process. When the cross-linking is accomplished, the cross-linking agent(s) may be entirely or partially linked to the hyaluronic acid or it may be degraded. Any remaining residuals of non-bound cross-linking agent can be removed after the cross-linking.

It is important to ensure that cross-linking does not occur until the preferred shape has been attained. This is advantageous for obtaining and maintaining a desired shape of the final product, since the shaping of the substrate is not limited by pre-existing cross-links, and all cross-links produced in the third step are directed to maintaining the desired shape of the product. It is preferred that the first two steps occur in the absence of a cross-linking agent, and that a cross-linking agent is added in the third cross-linking step. This provides a good control of the cross-linking since it only occurs in solid (precipitated) phase, and not in a dissolved phase and/or between method steps. It also ensures that the amount of cross-linking agent is tightly controlled and that the resulting products are homogenous in quality, since no cross-linking is reacted or lost in previous steps, e.g., during the precipitation step. Altogether, focusing the cross-linking, and in particular the addition of cross-linking agent, to the final step is useful to obtain a manufacturing process that is suitable for scaling up to an industrial scale and for providing products with homogenous quality.

The third liquid medium contains one or more cross-linking agent(s) that is polyfunctional, e.g., it has two or more reaction sites for forming covalent bonds to the hyaluronic acid molecules that are being cross-linked. It is preferred that the cross-linking agent(s) that is used in this third step is bifunctional, e.g., it has two reaction sites for forming covalent bonds to the hyaluronic acid molecules that are being cross-linked. Without being limited thereto, useful polyfunctional cross-linking agents include divinyl sulfone, multiepoxides and diepoxides, such as 1,4-butanediol diglycidyl ether (BDDE), 1,2-ethanediol diglycidyl ether (EDDE) and diepoxyoctane, preferably BDDE. It is desirable that the one or more polyfunctional cross-linking agent(s) provide ether cross-links. The present method advantageously provides ether cross-linked hyaluronic acid gel products which are stable and can readily be sterilized, e.g., autoclaved. Ester cross-links are less stable and will more easily be hydrolysed.

The cross-linking of the third method step is performed under precipitating conditions so that the shaped hyaluronic acid substrate is precipitated. In particular, the available surface of the hyaluronic acid molecules is in precipitated form due to the precipitating conditions. The third liquid medium contains an amount of one or more second organic solvent(s) giving precipitating conditions for hyaluronic acid, which amount and/or organic solvent(s) may be the same as or different to what was used in the second liquid medium of the second method step to precipitate the shaped hyaluronic acid substrate.

As detailed above, it shall be understood that the organic solvents are utilized for balancing and shifting the solubility of hyaluronic acid during the manufacturing method. The skilled person is well aware that other factors may have an impact on the limiting organic solvent(s) concentration for precipitation of hyaluronic acid, such as temperature, pH, ion strength and type of organic solvent(s). The limiting concentration for precipitation of hyaluronic acid under given conditions is well known or can easily be determined by a skilled person in the field.

Using this method, it is also possible to obtain shaped cross-linked hyaluronic acid products with a single cross-linking reaction in the third method step. Depending on the choice and number of cross-linking agents in the single reaction step, the resulting shaped product may be single cross-linked, e.g., containing essentially a single type of cross-links, preferably stable ether cross-links, or multiple cross-linked, e.g., containing at least two different types of cross-links, preferably including stable ether cross-links. It is surprising that a process with a single cross-linking reaction can achieve shaped products with such desirable properties.

Without being limited thereto, the organic solvents according to the invention can be selected from the group consisting of pentane, hexane, cyclohexane, 1,4-dioxane, N,N-dimethylformamide, N,N-dimethylacetamide, ethyl acetate, acetamide, diethyl ether, tetrahydrofurane, acetonitrile, methyl ethyl ketone, acetone, lower alkyl alcohols, e.g., methanol, ethanol, propanol, isopropanol and butanol, It is preferable that the organic solvents according to the invention are water-soluble. A preferred group of organic solvents is the lower alkyl alcohols. The term lower alkyl alcohol includes primary, secondary and tertiary alkyl alcohols having from one to six carbon atoms, e.g., C₁₋₆ alkyl alcohols. Specific examples of lower alkyl alcohols include methanol, ethanol, denatured spirit, n-propanol, isopropanol, n-butanol, isobutanol, and t-butanol. Preferred lower alkyl alcohols are methanol and ethanol, in particular ethanol, due to price, availability and easy handling.

It is suitable that the third liquid medium of the third method step is an aqueous medium, e.g., that it contains water to some extent. It is preferred that the third liquid medium, in addition to the cross-linking agent(s), contains 0-35 vol % water and 65-100 vol % of the second water-soluble organic solvent(s), preferably 20-35 vol % water and 65-80 vol % of the second water-soluble organic solvent(s).

The third liquid medium has a pH of 11.5 or higher, e.g., the cross-linking is performed at a pH of 11.5 or higher As the skilled person is well aware, the acidity of the hyaluronic acid starting material has to be taken into account to obtain a desired pH in the third liquid medium. This may be accomplished by addition of acids, bases or buffer systems with suitable buffering capacity. A preferred pH regulator is a strong base, such as sodium hydroxide. It has been found that a basic pH is advantageous when cross-linking the precipitated hyaluronic acid molecules. It is preferred that the third liquid medium of the third step has a pH of 13 or higher. In a liquid medium containing both water and organic solvent(s), the measured pH may differ from the theoretical pH due to the type of solvent(s) and amount of respective solvent(s). Therefore, the term apparent pH (pHₐₚₚ) is introduced to indicate the pH that is measured using standard pH measurement equipment under the given conditions. An accurate pH value can readily be determined by, e.g., titration.

The cross-linking in the third method step produces a shaped cross-linked hyaluronic acid product according to the invention. The shaped cross-linked hyaluronic acid product according to the invention is a gel, or a hydrogel. That is, it can be regarded as a water-insoluble, but substantially dilute cross-linked system of hyaluronic acid molecules when subjected to a liquid, typically an aqueous liquid. Since the cross-linking of the third method step is performed under precipitating conditions, both the shaped hyaluronic acid substrate and the shaped cross-linked product are precipitated. In particular, the available surface of both the shaped hyaluronic acid substrate and the shaped cross-linked product is in precipitated form due to the precipitating conditions of this third method step. The cross-linking reaction is allowed to proceed under suitable conditions until a desired amount of cross-linking agent(s) has reacted with the shaped hyaluronic acid substrate. The amount of cross-linking agent(s) that has bound to the hyaluronic acid can be quantified and reported as the degree of modification (MoD), e.g., the molar amount of bound cross-linking agent(s) relative to the total number of repeating HA disaccharide units. It is preferred that the cross-linking reaction is allowed to proceed until the degree of modification (MoD) of the cross-linked hyaluronic acid product is in the range of 1-40 cross-linking agent units per 1000 disaccharide units (0.1-4%), preferably 1-10 cross-linking agent units per 1000 disaccharide units (0.1-1%). The required reaction time is governed by several factors, such as hyaluronic acid concentration, cross-linking agent(s) concentration, temperature, pH, ion strength and type of organic solvent(s). These factors are all well known to the person skilled in the art, who easily can adjust these and other relevant factors and thereby provide suitable conditions to obtain a degree of modification in the range of 0.1-4% and verify the resulting product characteristics with respect to the degree of modification. The cross-linking of the third step occurs for at least 2 h, preferably at room temperature for at least 24 h.

Any residual non-bound cross-linking agent(s) can be removed when the shaped precipitated product is separated from the cross-linking medium. The shaped cross-linked hyaluronic acid product can be further purified by additional washing steps with a suitable washing liquid, e.g., water, methanol, ethanol, saline or mixtures and/or combinations thereof.

The manufacturing method according to the invention thus allows for a production of predefined physical forms, or structures, of the cross-linked hyaluronic acid products, such as a particle, a fibre, a string, a strand, a net, a film, a disc or a bead. Structures that are longitudinally extended, or rod-shaped, and have a ratio between their longitudinal extension and their largest lateral extension of 5:1 or higher, such as 10:1 or higher, e.g., 20:1 or higher, and optionally 100,000:1 or lower, such as 25,000:1 or lower, e.g., 100:1 or lower, are particularly useful as medical or cosmetic implants, because they can be dimensioned to avoid migration by having a sufficient length, and can at the same time readily be administered by injection through a needle due to the limited width. By way of example, a cross-linked hyaluronic acid product according to the invention with a single strand or fibre shape filling up a 20 ml syringe may have a thickness of 1 mm and a length of 25 m in swelled state, e.g., a ratio between its longitudinal extension and its largest lateral extension of 25,000:1.

The predefined structures can optionally be hollow or consist of multiple layers. The space created in a hollow predefined structure is optionally filled with HA, which may be modified, such as cross-linked or substituted with other compounds. One or more of the multiple layers in a predefined layer structure may consist of cross-linked or non-cross-linked HA, which may be chemically modified by substitution with other compounds. This may be accomplished by arranging the hyaluronic acid substrate in a desired shape in the first method step, e.g., by extrusion or molding, or in the second method step, e.g., by extrusion of the dissolved hyaluronic acid substrate into a precipitating medium, e.g., ethanol. The acquired shape can be maintained throughout the manufacturing method and in the final product.

Optionally, the manufacturing method further involves a fourth step of subjecting the shaped precipitated cross-linked hyaluronic acid product to non-precipitating conditions. That is, the method is in certain embodiments comprising four steps, or alternatively consisting of four steps. This typically involves subjecting the shaped cross-linked hyaluronic acid product to a liquid medium and allowing it to return to non-precipitated state. The liquid medium is typically water, saline or mixtures and/or combinations thereof, optionally with non-precipitating concentrations of an organic solvent, e.g., methanol or ethanol. Due to the cross-linking, the resulting shaped hyaluronic acid product is a continuous network of interconnected and entangled hyaluronic acid chains which under non-precipitating conditions absorbs liquid (swells) and forms a gel. The swelling can be allowed to proceed until the gel is fully swollen and no further liquid can be absorbed, or it can be interrupted at an earlier stage to obtain a partially swollen gel. A partially swollen gel can be useful as an intermediate for further processing of the gel, for instance further mechanical production of gel structures of a desired size and shape can be performed. By way of example, a film can be cut into particles, slices or pieces, gel fibers can be cut into shorter fragments, well defined irregular shapes can be designed from a film, etc. The cross-linked HA fibers, strings or strands can also be woven together to form a net or a film after completed cross-linking, before or after drying. It may also be convenient to use a partially swollen shaped gel product during implantation thereof at a desired site to facilitate administration and minimize patient discomfort and to enhance the lifting capacity by use of the remaining swelling capacity.

When the shaped gel product is subjected to non-precipitating conditions in an excess of liquid, it is also possible to determine its maximum swelling degree, or inversely its minimum hyaluronic acid concentration (Cₘᵢₙ), i.e., the hyaluronic acid concentration when the gel product is fully swollen. Using the manufacturing method according to the invention, it is possible to obtain a swelling degree of 4-300 mL per g hyaluronic acid, and preferably 15-180 mL per g hyaluronic acid. This implies Cₘᵢₙ values in the range of 0.3-25% (w/v), preferably 0.6-7% (w/v), corresponding to 3-250 mg/g, preferably 6-70 mg/g. It is highly advantageous that the desired swelling degree (or Cₘᵢₙ value) can be achieved with a minimal degree of modification, but the traditional way of regulating the swelling degree is by means of varying the degree of modification. The present manufacturing method therefore provides a new concept for regulating the swelling capacity of a shaped gel product, which surprisingly enables production of firm shaped gels with a uniquely high Cₘᵢₙ value (low swelling degree) in relation to the low degree of modification of the gel.

The modification efficiency (MoE) is a measure of the ratio between the minimum HA concentration (Cₘᵢₙ), or rigidity/strength, of a gel and its degree of chemical modification by cross-linking agent(s). Using the manufacturing method according to the present invention, it is possible to obtain a cross-linked hyaluronic acid product having a modification efficiency of 10 or higher, preferably in the range of 10-200, such as 20-150 or 20-190. Without desiring to be limited to any specific theory, it is contemplated that the beneficial properties of the gel are the result of a surprisingly high degree of effective cross-linking, e.g., a high degree of the bound cross-linking agent(s) (cross-linker ratio, typically 0.35 or 35% or higher, such as 40% or higher or even 50% or higher) is in fact bound to the hyaluronic acid at two (or more) sites, in combination with effective positioning of the cross-links for the desired purpose, and probably an extremely high degree of retained entanglement. In contrast to what a skilled person would expect from the low degree of modification of the resulting hyaluronic acid product, the method according to the invention surprisingly provides a gel with high rigidity/strength. Under any circumstances, the method according to the invention provides a useful way of further regulating the swelling degree in relation to the degree of modification. The method is also very suitable for continuous operation, which is advantageous for large-scale production.

Optionally, the manufacturing method also involves a final step of isolating the cross-linked hyaluronic acid product. That is, the method is in certain embodiments comprising four or five steps, or alternatively consisting of four or five steps. Depending on whether the product is held under precipitating conditions or has been subjected to non-precipitating conditions, this step may involve isolating the product in precipitated form or in non-precipitated form. The isolated, precipitated or non-precipitated, product can then be subjected to sterilization so as to obtain a sterile cross-linked hyaluronic acid product.

If desired, other substances, such as local anesthetics (e.g., lidocaine hydrochloride) anti-inflammatory drugs, antibiotics and other suitable supportive medications, e.g., bone growth factors or cells, may be added after the cross-linked hyaluronic acid product has been obtained.

According to one aspect, the invention provides a shaped cross-linked hyaluronic acid product. According to one embodiment, the product is manufactured, or can be manufactured, by the manufacturing method of the invention. The shaped cross-linked hyaluronic acid product according to the invention is a gel, or a hydrogel. That is, it can be regarded as a water-insoluble, but substantially dilute cross-linked system of hyaluronic acid molecules when subjected to a liquid, typically an aqueous liquid. The gel is mostly liquid by weight and can contain, e.g., 90-99.9% water, but it behaves like a solid due to a three-dimensional cross-linked hyaluronic acid network within the liquid. Due to its significant liquid content, the shaped gel is structurally flexible and similar to natural tissue, which makes it very useful as a scaffold in tissue engineering and for tissue augmentation. It is the cross-links and their attachment positions at the hyaluronic acid molecules that, together with the natural entanglement of the hyaluronic acid chains, give the gel its structure and properties, which are intimately related to its swelling degree.

The amount of attached cross-linking agent(s) can be quantified by and reported as the degree of modification (MoD), i.e., the molar amount of bound cross-linking agent(s) relative to the total number of repeating HA disaccharide units. It is preferred that the cross-linked hyaluronic acid product according to the invention has a degree of modification of 1-40 cross-linking agent units per 1000 disaccharide units (0.1-4%), preferably 1-10 cross-linking agent units per 1000 disaccharide units (0.1-1%). The effectiveness of the cross-linking reaction is shown by the amount of attached cross-linking agent(s) that is connected in (at least) two ends to one (or more) hyaluronic acid chains and is reported as the cross-linker ratio (CrR). It is preferable that the product according to the invention has a cross-linker ratio of 35% or higher, preferably 40% or higher, more preferably 50% or higher, such as in the ranges of 35-80%, 40-80% and 50-80%. These products consequently have a low number of cross-linking agents that do not provide effective cross-links in the product. The high cross-linker ratios allow for a surprisingly low total degree of modification in relation to the gel strength, which in turn is advantageous to ensure high biocompatibility.

### Hydrogel Production Method 5

In some aspects, the hydrogels are created by intermingling different hydrogels. In some aspects, the different hydrogels are different in that they comprise a different degree of crosslinking, comprise a different swelling factor, and/or comprise a different G′ (Pa) (the measure of elastic modulus). In some aspects, the different hydrogels, once intermingled, are subject further crosslinking. In some aspects, the different hydrogels, prior to intermingling, are formed into various sizes of particles. In some aspects, this results in both differences in (1) particle size and (2) crosslinking degree, clastic modulus, and/or swelling factor. In some aspects, intermingling hydrogels may include blending different, separately-prepared hydrogels. In some aspects, intermingling hydrogels may include cross-linking separately-prepared hydrogels.

In some aspects, a hydrogel composition comprises at least 2 different hydrogels. In some aspects, a hydrogel composition comprises at least 3 different hydrogels. In some aspects, a hydrogel composition comprises at least 4 different hydrogels. In some aspects, a hydrogel composition comprises at least 5 different hydrogels. In some aspects, a hydrogel composition comprises at least 6 different hydrogels. In some aspects, each of the hydrogels differ by (1) particle size and/or (2) crosslinking degree, elastic modulus, and/or swelling factor.

In some aspects, a hydrogel composition comprises a first hydrogel having a first crosslinking degree, a second hydrogel having a second crosslinking degree, and/or a third hydrogel having a third crosslinking degree; wherein the first, second, and/or third hydrogels are intermingled and formed into particles of the same or different size.

### Temperature, Timing, and Crosslinking Conditions for Modulating Hydrogel Properties

The following time, temperature, cross-linking conditions, and hydrogel properties are intended to apply to any hydrogel production methods discussed in this disclosure and are not intended to be limiting in any aspect.

In some aspects, the crosslinking with the GAG and the crosslinker occurs at about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C.

In some aspects, the crosslinking with the GAG and the crosslinker occurs at about 1° C. to about 75° C., about 1° C. to about 65° C., about 1° C. to about 55° C., about 1° C. to about 40° C., about 1° C. to about 25° C., about 1° C. to about 20° C., about 1° C. to about 15° C., about 1° C. to about 10° C., about 1° C. to about 5° C., 2° C. to about 75° C., about 2° C. to about 65° C., about 2° C. to about 55° C., about 2° C. to about 40° C., about 2° C. to about 25° C., about 2° C. to about 20° C., about 2° C. to about 15° C., about 2° C. to about 10° C., about 2° C. to about 5° C., 3º° C. to about 75° C., about 3° C. to about 65° C., about 3° C. to about 55° C., about 3° C. to about 40° C., about 3° C. to about 25° C., about 3° C. to about 20° C., about 3° C. to about 15° C., about 3° C. to about 10° C., about 3° C. to about 5° C., 4° C. to about 75° C., about 4° C. to about 65° C., about 4° C. to about 55° C., about 3° C. to about 40° C., about 4° C. to about 25° C., about 4° C. to about 20° C., about 4° C. to about 15° C., about 4° C. to about 10° C., about 4° C. to about 5° C., 5° C. to about 75° C., about 5° C. to about 65° C., about 5° C. to about 55° C., about 5° C. to about 40° C., about 5° C. to about 25° C., about 5° C. to about 20° C., about 5° C. to about 15° C., about 5° C. to about 10° C., 7° C. to about 75° C., about 7° C. to about 65° C., about 7° C. to about 55° C., about 7° C. to about 40° C., about 7° C. to about 25° C., about 7° C. to about 20° C., about 7° C. to about 15° C., about 7° C. to about 10° C., 9° C. to about 75° C., about 9° C. to about 65° C., about 9° C. to about 55° C., about 9° C. to about 40° C., about 9° C. to about 25° C., about 9° C. to about 20° C., about 9° C. to about 15° C., and about 9° C. to about 10° C.

In some aspects, the crosslinking with the GAG and the crosslinker occurs at less than 0° C., less than 1° C., less than 2° C., less than 3° C., less than 4° C., less than 5° C., less than 6° C., less than 7° C., less than 8° C., less than 9° C., less than 10° C., less than 11° C., less than 12° C., less than 13° C., less than 14° C., less than 15° C., less than 16° C., less than 17° C., less than 18° C., less than 19° C., less than 20° C., less than 21° C., less than 22° C., less than 23° C., less than 24° C., or less than 25° C.

In some aspects, the crosslinking with the GAG and the crosslinker occurs at greater than 25° C., greater than 26° C., greater than 27° C., greater than 28° C., greater than 29° C., greater than 30° C., greater than 31° C., greater than 32° C., greater than 33° C., greater than 34° C., greater than 35° C., greater than 36° C., greater than 37° C., greater than 38° C., greater than 39° C., greater than 40° C., greater than 41° C., greater than 42° C., greater than 43° C., greater than 44° C., greater than 45° C., greater than 46° C., greater than 47° C., greater than 48° C., greater than 49° C., greater than 50° C., greater than 51° C., greater than 52° C., greater than 53° C., greater than 54° C., greater than 55° C., greater than 56° C., greater than 57° C., greater than 58° C., greater than 59° C., greater than 60° C., greater than 65° C., greater than 70° C., or greater than 75° C.

In some aspects, the crosslinking with the GAG and the crosslinker occurs at 25° C. to 75° C., 25° C. to 70° C., 25° C. to 65° C., 25° C. to 60° C., 25° C. to 50° C., 25° C. to 45° C., 25° C. to 40° C., 25° C. to 35° C., 25° C. to 30° C., 30° C. to 75° C., 30° C. to 70° C., 30° C. to 65° C., 30° C. to 60° C., 30° C. to 50° C., 30° C. to 45° C., 30° C. to 40° C., 30° C. to 35° C., 35° C. to 75° C., 35° C. to 70° C., 35° C. to 65° C., 35° C. to 60° C., 35° C. to 50° C., 35° C. to 45° C., 35° C. to 40° C., 40° C. to 75° C., 40° C. to 70° C., 40° C. to 65° C., 40° C. to 60° C., 40° C. to 50° C., 40° C. to 45° C., 45° C. to 75° C., 45° C. to 70° C., 45° C. to 65° C., 45° C. to 60° C., 45° C. to 50° C., 50° C. to 75° C., 50° C. to 70° C., 50° C. to 65° C., 50° C. to 60° C., 50° C. to 55° C., 55° C. to 75° C., 55° C. to 70° C., 55° C. to 65° C., 55° C. to 60° C., 60° C. to 75° C., 60° C. to 70° C., 60° C. to 65° C., 65° C. to 75° C., 65° C. to 70° C., or 70° C. to 75° C.

In some aspects, the crosslinking with the GAG and the crosslinker occurs at about 25° C. to about 75° C., about 25° C. to about 70° C., about 25° C. to about 65° C., about 25° C. to about 60° C., about 25° C. to about 50° C., about 25° C. to about 45° C., about 25° C. to about 40° C., about 25° C. to about 35° C., about 25° C. to about 30° C., about 30° C. to about 75° C., about 30° C. to about 70° C., about 30° C. to about 65° C., about 30° C. to about 60° C., about 30° C. to about 50° C., about 30° C. to about 45° C., about 30° C. to about 40° C., about 30° C. to about 35° C., about 35° C. to about 75° C., about 35° C. to about 70° C., about 35° C. to about 65° C., about 35° C. to about 60° C., about 35° C. to about 50° C., about 35° C. to about 45° C., about 35° C. to about 40° C., about 40° C. to about 75° C., about 40° C. to about 70° C., about 40° C. to about 65° C., about 40° C. to about 60° C., about 40° C. to about 50° C., about 40° C. to about 45° C., about 45° C. to about 75° C., about 45° C. to about 70° C., about 45° C. to about 65° C., about 45° C. to about 60° C., about 45° C. to about 50° C., about 50° C. to about 75° C., about 50° C. to about 70° C., about 50° C. to about 65° C., about 50° C. to about 60° C., about 50° C. to about 55° C., about 55° C. to about 75° C., about 55° C. to about 70° C., about 55° C. to about 65° C., about 55° C. to about 60° C., about 60° C. to about 75° C., about 60° C. to about 70° C., about 60° C. to about 65° C., about 65° C. to about 75° C., about 65° C. to about 70° C., or about 70° C. to about 75° C.

In some aspects, the crosslinking with the GAG and the crosslinker occurs for about 30 minutes, about 40 minutes, about 50 minutes, about 60 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 30 hours, about 35 hours, about 40 hours, about 45 hours, about 50 hours, about 55 hours, about 60 hours, about 65 hours, about 70 hours, about 75 hours, about 80 hours, about 85 hours, about 90 hours, about 95 hours, about 100 hours, about 120 hours, about 140 hours, about 160 hours, about 180 hour, or about 200 hours.

In some aspects, the crosslinking with the GAG and the crosslinker occurs for at least about 30 minutes, at least about 40 minutes, at least about 50 minute, at least about 60 minutes, at least about 2 hours, at least about 3 hours, at least about 4 hours, at least about 5 hours, at least about 6 hours, at least about 7 hours, at least about 8 hours, at least about 9 hours, at least about 10 hours, at least about 15 hours, at least about 20 hours, at least about 24 hours, at least about 25 hours, at least about 30 hours, at least about 35 hours, at least about 36 hours, at least about 40 hours, at least about 50 hours, at least about 60 hours at least about 80 hour, at least about 100 hours, at least about 120 hours, at least about 140 hours, at least about 160 hours, at least about 180 hours, or at least about 200 hours.

In some aspects, the swelling of the GAG occurs for about 30 minutes, about 40 minutes, about 50 minutes, about 60 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 30 hours, about 35 hours, about 40 hours, about 45 hours, about 50 hours, about 55 hours, about 60 hours, about 65 hours, about 70 hours, about 75 hours, about 80 hours, about 85 hours, about 90 hours, about 95 hours, about 100 hours, about 120 hours, about 140 hours, about 160 hours, about 180 hour, or about 200 hours.

In some aspects, the swelling of the GAG occurs for at least about 30 minutes, at least about 40 minutes, at least about 50 minute, at least about 60 minutes, at least about 2 hours, at least about 3 hours, at least about 4 hours, at least about 5 hours, at least about 6 hours, at least about 7 hours, at least about 8 hours, at least about 9 hours, at least about 10 hours, at least about 15 hours, at least about 20 hours, at least about 24 hours, at least about 25 hours, at least about 30 hours, at least about 35 hours, at least about 36 hours, at least about 40 hours, at least about 50 hours, at least about 60 hours at least about 80 hour, at least about 100 hours, at least about 120 hours, at least about 140 hours, at least about 160 hours, at least about 180 hours, or at least about 200 hours.

In some aspects, each of the swelling or crosslinking time points or ranges of time disclosed in the present application are contemplated as being performed at each of the temperatures or ranges of temperatures disclosed herein.

In some aspects, the swelling of the crosslinked GAG molecules occurs until the gel is at a concentration of about 10 mg/g of the solution, about 12 mg/g of the solution, about 14 mg/g of the solution, about 16 mg/g of the solution, about 18 mg/g of the solution, about 20 mg/g of the solution, about 22 mg/g of the solution, about 24 mg/g of the solution, about 26 mg/g of the solution, about 28 mg/g of the solution, about 30 mg/g of the solution, about 32 mg/g of the solution, about 34 mg/g of the solution, about 36 mg/g of the solution, about 38 mg/g of the solution, or about 40 mg/g of the solution.

In some aspects, the swelling of the crosslinked GAG molecules occurs until the gel is at concentration of about 10 mg/g of the solution to about 40 mg/g of the solution, about 12 mg/g of the solution to about 40 mg/g of the solution, about 14 mg/g of the solution to about 40 mg/g of the solution, about 16 mg/g of the solution to about 40 mg/g of the solution, about 18 mg/g of the solution to about 40 mg/g of the solution, about 20 mg/g of the solution to about 40 mg/g of the solution, about 22 mg/g of the solution to about 40 mg/g of the solution, about 24 mg/g of the solution to about 40 mg/g of the solution, about 10 mg/g of the solution to about 35 mg/g of the solution, about 10 mg/g of the solution to about 30 mg/g of the solution, about 10 mg/g of the solution to about 25 mg/g of the solution, about 10 mg/g of the solution to about 20 mg/g of the solution, about 10 mg/g of the solution to about 18 mg/g of the solution, about 10 mg/g of the solution to about 16 mg/g of the solution, about 10 mg/g of the solution to about 14 mg/g of the solution, or about 10 mg/g of the solution to about 12 mg/g of the solution.

In some aspects, once the GAG hydrogel is swelled to the desired concentration, it is dehydrated. In some aspects, once the GAG hydrogel is passed through one or more mechanisms to reduce the hydrogel to a plurality of beads or microbeads.

In some aspects, the microbeads are packaged into a container comprising about 1000 beads per ml, about 2000 beads per ml, about 3000 beads per ml, about 4000 beads per ml, about 5000 beads per ml, about 6000 beads per ml, about 7000 beads per ml, about 8000 beads per ml, about 9000 beads per ml, about 10000 beads per ml, about 15000 beads per ml, about 20000 beads per ml, about 25000 beads per ml, about 30000 beads per ml.

In some aspects, the alkaline conditions comprise a pH of about 7.5, about 8, about 8.5, about 9, about 9.5, about 10, about 10.5, about 11, about 11.5, or about 12. In some aspects, the alkaline conditions comprise a pH of about 7.5 to about 12, about 8 to about 11, about 8.5 to about 10.5, or about 9 to about 10. In some aspects, the alkaline conditions are created with a hydroxide. In some aspects, the alkaline conditions are created with a metal hydroxide. In some aspects, the alkaline conditions are created with sodium hydroxide. In some aspects, the alkaline conditions are created with an organic hydroxide.

In some aspects, the hydroxide concentration during crosslinking is about 0.5 w/w %, about 0.55 w/w %, about 0.6 w/w %, about 0.65 w/w %, about 0.7 w/w %, about 0.75 w/w %, about 0.8 w/w %, about 0.85 w/w %, about 0.9 w/w %, about 0.95 w/w %, or about 1 w/w %.

In some aspects, the hydroxide concentration during crosslinking is about 0.5 w/w % to about 10 w/w %, about 0.5 w/w % to about 9 w/w %, about 0.5 w/w % to about 8 w/w %, about 0.5 w/w % to about 7 w/w %, about 0.5 w/w % to about 6 w/w %, about 0.5 w/w % to about 5 w/w %, about 0.5 w/w % to about 4 w/w %, about 0.5 w/w % to about 3 w/w %, about 0.5 w/w % to about 2 w/w %, about 0.55 w/w % to about 1 w/w %, about 0.7 w/w % to about 1 w/w %, about 0.8 w/w % to about 1 w/w %, about 0.9 w/w % to about 1 w/w %, about 0.5 w/w % to about 0.9 w/w %, about 0.5 w/w % to about 0.8 w/w %, about 0.5 w/w % to about 0.7 w/w %, or about 0.5 w/w % to about 0.6 w/w %.

In some aspects, the alkaline conditions are neutralized after crosslinking. In some aspects, the alkaline conditions are neutralized with the addition of a base. In some aspects, the alkaline conditions are neutralized by diluting the solution comprising the crosslinked GAG/crosslinked HA.

In some aspects, the hyaluronic acid or other GAG concentration added to the crosslinking reaction is about 5 w/w %, about 6 w/w %, about 7 w/w %, about 8 w/w %, about 9 w/w %, about 10 w/w %, about 11 w/w %, about 12 w/w %, about 13 w/w %, about 14 w/w %, about 15 w/w %, about 16 w/w %, about 17 w/w %, about 18 w/w %, about 19 w/w %, about 20 w/w %, about 21 w/w %, about 22 w/w %, about 23 w/w %, about 24 w/w %, about 25 w/w %, about 26 w/w %, about 27 w/w %, about 28 w/w %, about 29 w/w %, about 30 w/w %, about 35 w/w %, about 40 w/w %, about 45 w/w %, about 50 w/w %, about 55 w/w %, about 60 w/w %, about 70 w/w %, or about 75 w/w %, or about 80 w/w %,

In some aspects, the hyaluronic acid or other GAG concentration added to the crosslinking reaction is about 5 w/w % to about 80 w/w %, about 5 w/w % to about 75 w/w %, about 5 w/w % to about 60 w/w %, about 5 w/w % to about 50 w/w %, about 5 w/w % to about 40 w/w %, about 5 w/w % to about 30 w/w %, about 5 w/w % to about 25 w/w %, about 5 w/w % to about 20 w/w %, about 5 w/w % to about 15 w/w %, about 5 w/w % to about 10 w/w %, about 15 w/w % to about 80 w/w %, about 20 w/w % to about 80 w/w %, about 25 w/w % to about 80 w/w %, about 30 w/w % to about 80 w/w %, about 40 w/w % to about 80 w/w %, about 50 w/w % to about 80 w/w %, about 60 w/w % to about 80 w/w %, or about 70 w/w % to about 80 w/w %.

In some aspect, the solution in which the swelling occurs is an aqueous solution. In some aspects, the aqueous solution comprises a dissolved salt. In some aspects, the aqueous solution comprises dissolved sodium chloride. In some aspects, the aqueous solution is a buffered solution. In some aspects, the aqueous solution comprises a phosphate buffer. In some aspects, the aqueous solution is 0.9% sodium chloride.

In some aspects, the hydrogel product crosslinked at one of the crosslinking temperatures described herein exhibits a degradation rate for the GAG (e.g., HA) chains, during crosslinking, of about 500× less or greater than a degradation rate of GAG chains (e.g., HA chains) during crosslinking at a temperature of about 5° C., about 10° C., about 25°, about 40° C., about 45° C. or about 50° C. In some aspects, the hydrogel product crosslinked at one of the crosslinking temperatures described herein exhibits a degradation rate for the GAG (e.g., HA) chains, during crosslinking, of about 500×, about 450×, about 400×, about 350×, about 300×, about 250×, about 200×, about 150×, about 100×, about 90×, about 80×, about 70×, about 60×, about 50×, about 40×, about 30×, about 20×, about 10×, about 5×, about 4×, about 3×, about 2×, or about 1× less than a degradation rate of GAG chains (e.g., HA chains) during crosslinking at a temperature of about 5° C., about 10° C., about 25°, about 40° C., about 45° C. or about 50° C.

In some aspects, the hydrogel product crosslinked at one of the crosslinking temperatures described herein exhibits a decrease or increase in firmness as compared to a control hydrogel crosslinked at a temperature of about 5° C., about 10° C., about 25°, about 40° C., about 45° C. or about 50° C. In some aspects, the hydrogel product crosslinked at one of the crosslinking temperatures described herein exhibits a decrease or increase in firmness of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 200%, about 500%, about 1000%, or about 5000% compared to a control hydrogel crosslinked at a temperature of about 25°. In some aspects, the hydrogel product crosslinked at one of the crosslinking temperatures described herein exhibits a decrease or increase in firmness of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 500%, at least about 1000%, or at least about 5000% compared to a control hydrogel crosslinked at a temperature of about 5° C., about 10° C., about 25°, about 40° C., about 45° C. or about 50° C.

In some aspects, the hydrogel product crosslinked at one of the crosslinking temperatures described herein exhibits a hydrogel comprising shorter or longer GAG chains as compared to a control hydrogel crosslinked at a temperature of about 5° C., about 10° C., about 25°, about 40° C., about 45° C. or about 50° C. In some aspects, the hydrogel product crosslinked at one of the crosslinking temperatures described herein exhibits a decrease or increase in the GAG chains of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 200%, about 500%, about 1000%, or about 5000% compared to a control hydrogel crosslinked at a temperature of about 5° C., about 10° C., about 25°, about 40° C., about 45° C. or about 50° C. In some aspects, the hydrogel product crosslinked at one of the crosslinking temperatures described herein exhibit a decrease or increase in the GAG chains of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 500%, at least about 1000%, or at least about 5000% compared to a control hydrogel crosslinked at a temperature of about 5° C., about 10° C., about 25°, about 40° C., about 45° C. or about 50° C.

In some aspects, the hydrogel product crosslinked at one of the crosslinking temperatures described herein exhibits an increase or decrease of the elastic modulus of about 500× as compared to a hydrogel crosslinked at a temperature of about 5° C., about 10° C., about 25°, about 40° C., about 45° C. or about 50° C. In some aspects, the hydrogel product crosslinked at one of the crosslinking temperatures described herein exhibits an increase or decrease of the clastic modulus of about 500×, about 450×, about 400×, about 350×, about 300×, about 250×, about 200×, about 150×, about 100×, about 90×, about 80×, about 70×, about 60×, about 50×, about 40×, about 30×, about 20×, about 10×, about 5×, about 4×, about 3×, about 2×, or about 1× less than a hydrogel crosslinked at a temperature of about 5° C., about 10° C., about 25°, about 40° C., about 45° C. or about 50° C.

In some aspects, the crosslinked GAG is crosslinked via ether bonds to the crosslinker. In some aspects, the crosslinked GAG is crosslinked via ester bonds to the crosslinker.

In some aspects, the GAG is a sulfated or non-sulfated GAG such as hyaluronan, chondroitin sulphate, heparan sulphate, heparosan, heparin, dermatan sulphate and keratan sulphate. In some aspects, the GAG is hyaluronic acid, chondroitin or chondroitin sulfate. In one aspect, the GAG is hyaluronic acid. In some aspects, the GAG is a native GAG. In some aspects, the GAG is a naturally occurring GAG. In some aspects, the GAG is used in its native state (e.g., the chemical structure of the GAG has not been altered or modified by addition of functional groups or the like). Using the GAG in its native state is preferred because this will afford a crosslinked structure more closely resembling the natural molecules, which conserves the native properties and effects of the GAG itself, and can minimize the immune response when the crosslinked GAG is introduced into the body.

In some aspects, the GAGs are covalently crosslinked. In some aspects, the covalently crosslinked GAG molecules consist of, or essentially consist of carbohydrate type structures or derivatives thereof. In some aspects, the crosslinked GAGs or hydrogels are free of, or essentially free of synthetic non-carbohydrate structures or linkers. This can be achieved by using a GAG in its native state together with a crosslinker which comprises, consists of, or essentially consist of carbohydrate type structures or derivatives thereof. In some aspects, functional groups of the crosslinker are covalently bound directly to carboxyl groups of the GAG.

In some aspects, the crosslinked GAG comprises crosslinks between the GAG molecule chains, which creates a continuous network of GAG molecules held together by covalent crosslinks.

In some aspects, the crosslinked GAGs form a gel or hydrogel-water-insoluble, but substantially dilute crosslinked system of GAGs when subject to liquid, typically an aqueous liquid.

In some aspects, the process of preparing a hydrogel product comprising crosslinked glycosaminoglycan molecules, comprises, consists of, or consists essentially of: (a) providing a solution of glycosaminoglycan molecules; (b) activating carboxyl groups on the glycosaminoglycan molecules with a coupling agent to form activated, glycosaminoglycan molecules; and (c) crosslinking the activated glycosaminoglycan molecules via their activated carboxyl groups using a di- or multinucleophile functional crosslinker comprising a spacer group.

In some aspects, the GAGs are crosslinked by covalent bonds, such as amide bonds, typically using an activating agent for the carboxyl groups on the GAG molecule backbone and a di- or multinucleophile functional crosslinker comprising a spacer group. In some aspects, crosslinking of the GAGs can be achieved by mild and efficient routes resulting in high yields with minimal degradation of the GAG molecules.

In some aspects, the di- or multinucleophile functional crosslinker contains a spacer group with at least two nucleophile functional groups attached thereto. In some aspects, the at least two nucleophile functional groups are separated by the spacer group.

In some aspects, the di- or multinucleophile functional crosslinker comprises two or more functional groups capable of reacting with functional carboxyl groups of the GAG, resulting in the formation of covalent bonds, such as amide bonds. In some aspects, the nucleophile functional groups are capable of reacting with carboxyl groups on the glycosaminoglycan molecule to form amide bonds. In some aspects, the nucleophile functional groups of crosslinker are selected from the group consisting of primary amine, hydrazine, hydrazide, carbazate, semi-carbazide, thiosemicarbazide, thiocarbazate, and aminoxy groups. In some aspects, crosslinker molecules have been modified by introduction of two or more nucleophile functional groups.

In some aspects, the di- or multinucleophile functional crosslinker include homo- or heterobifunctional primary amines, hydrazines, hydrazides, carbazates, semi-carbazides, thiosemicarbazides, thiocarbazates and aminoxy groups.

In some aspects, the activation step and the crosslinking step occur simultaneously. In some aspects, the activation step occurs prior to and separately from the crosslinking step.

In some aspects, a step subsequent to crosslinking comprises providing particles of the crosslinked GAG molecule, having an average size in the range of 0.01-5 mm, preferably 0.1-0.8 mm.

In some aspects, the particles are between 20 to 800 μm in size. In some aspects, the particles are between about 100 to about 500 μm in size. In some aspect, this size may be length, diameter, or width. In general, this refers to diameter. In some aspects, the particles are between 20 to 800 μm, between 20 to 700 μm, between 20 to 600 μm, between 20 to 500 μm, between 20 to 400 μm, between 20 to 300 μm, between 20 to 200 μm, between 100 to 800 μm, between 100 to 700 μm, or between 100 to 300 μm in size. In some embodiments, the particles are less than 1 cm, less than 1 mm, or less than 100 μm in size in size.

In some aspects, the coupling agent is a peptide coupling reagent. In some aspects the coupling reagent is selected from 4-(4,6-dimethoxy-1,3,5-triazin-2-yl)-4-methylmorpholinium chloride (DMTMM) and 2-chloro-4,6-dimethoxy-1,3,5-triazine (CDMT). A preferred triazine-based peptide coupling reagent is DMTMM. Other preferred peptide coupling reagent are carbodiimide coupling reagents, preferably N-(3-dimethylaminopropyl)-N′-ethylcarbodiimide (EDC) combined with N-hydroxysuccinimide (NHS).

In some aspects, crosslinking of the activated GAG molecules occurs via their carboxyl groups using a crosslinker. In some aspects, the crosslinker is a di- or multinucleophile functional crosslinker comprising a spacer group. In some aspects, the crosslinker connects the GAG chains to each other via carboxyl groups on the GAG backbone. In some aspects, the spacer group may be a hyaluronic acid-crosslinker residue. By the term “residue” is meant here that the structure of the compound is similar but not identical to the parent compounds hyaluronic acid. The structure of the residue may differ from the structure of the parent compound in that it has been provided with two or more nucleofile functional groups and optionally covalently linked via said nucleophile functional groups carboxyl groups on the GAG backbone.

According to a related aspect, the present invention also provides use of the hydrogel product as a medicament, such as in the treatment of soft tissue disorders. There is provided a method of treating a patient suffering from a soft tissue disorder by administering to the patient a therapeutically effective amount of the hydrogel product. There is also provided a method of providing corrective or aesthetic treatment to a patient by administering to the patient a therapeutically effective amount of the hydrogel product.

In some aspects, the hydrogel contains mostly liquid by weight and can contain 90-99.9%, water, but it behaves like a solid due to a three-dimensional crosslinked GAG molecule network within the liquid. Due to its significant liquid content, the hydrogel is structurally flexible and similar to natural tissue, which makes it very useful as a scaffold in tissue engineering and for tissue augmentation. It is also useful for treatment of soft tissue disorder and for corrective or esthetic treatment. In some aspects, the hydrogel is used as an injectable formulation.

The methods disclosed herein are methods of using the injectable compositions for reparative or plastic surgery, esthetic dermatology, facial contouring, body contouring, and gingival augmentation. In some aspects, the compositions are freeze-dried or lyophilized. In some aspects, the compositions comprise a hydrogel comprising an aqueous solution.

In some aspects, the suitable GAG concentration is 10 to 50 mg/mL, 10 to 45 mg/mL, 10 to 40 mg/mL, 10 to 35 mg/mL, 10 to 30 mg/mL, 10 to 25 mg/mL, 10 to 20 mg/mL, 10 to 15 mg/mL, 15 to 40 mg/mL, 15 to 40 mg/mL, 15 to 35 mg/mL, 15 to 30 mg/mL, 15 to 25 mg/mL, 15 to 20 mg/mL, 20 to 50 mg/mL, 20 to 45 mg/mL, 20 to 40 mg/mL, 20 to 35 mg/mL, 20 to 30 mg/mL, 20 to 25 mg/mL, 25 to 50 mg/mL, 25 to 45 mg/mL, 25 to 40 mg/mL, 25 to 35 mg/mL, 25 to 30 mg/mL, 30 to 50 mg/mL, 30 to 45 mg/mL, 30 to 40 mg/mL, 30 to 35 mg/mL, 35 to 50 mg/mL, 35 to 45 mg/mL, 35 to 40 mg/mL, 40 to 50 mg/mL, or 40 to 45 mg/mL.

In some aspects, the suitable GAG concentration is about 10 to about 50 mg/mL, about 10 to about 45 mg/mL, about 10 to about 40 mg/mL, about 10 to about 35 mg/mL, about 10 to about 30 mg/mL, about 10 to about 25 mg/mL, about 10 to about 20 mg/mL, about 10 to about 15 mg/mL, about 15 to about 40 mg/mL, about 15 to about 40 mg/mL, about 15 to about 35 mg/mL, about 15 to about 30 mg/mL, about 15 to about 25 mg/mL, about 15 to about 20 mg/mL, about 20 to about 50 mg/mL, about 20 to about 45 mg/mL, about 20 to about 40 mg/mL, about 20 to about 35 mg/mL, about 20 to about 30 mg/mL, about 20 to about 25 mg/mL, about 25 to about 50 mg/mL, about 25 to about 45 mg/mL, about 25 to about 40 mg/mL, about 25 to about 35 mg/mL, about 25 to about 30 mg/mL, about 30 to about 50 mg/mL, about 30 to about 45 mg/mL, about 30 to about 40 mg/mL, about 30 to about 35 mg/mL, about 35 to about 50 mg/mL, about 35 to about 45 mg/mL, about 35 to about 40 mg/mL, about 40 to about 50 mg/mL, or about 40 to about 45 mg/mL.

In some aspects, hyaluronic acid encompasses all variants and combinations of variants of hyaluronic acid, hyaluronate, or hyaluronan—of various chain lengths and charge states, as well as various chemical modifications, including crosslinking.

In some aspects, hyaluronic acid encompasses the various hyaluronate salts of hyaluronic acid with various counter ions, such as sodium hyaluronate. In some aspects, various modifications of hyaluronic acid are also encompassed by recitation of hyaluronic acid, such as oxidation, e.g., oxidation of —CH₂OH groups to —CHO and/or —COOH; periodate oxidation of vicinal hydroxyl groups, which may be followed by reduction, e.g., reduction of —CHO to —CH₂OH or coupling with amines to form imines followed by reduction to secondary amines; sulphation; deamidation, which may be followed by deamination or amide formation with new acids; esterification; crosslinking; substitutions with various compounds, e.g., using a crosslinking agent or a carbodiimide assisted coupling; including coupling of different molecules, such as proteins, peptides and active drug components, to hyaluronic acid; and deacetylation. In some aspects, hyaluronic acid may be further modified by isourea, hydrazide, bromocyan, monocpoxide, and monosulfone couplings.

In some aspects, hyaluronic acid may be obtained from various sources of animal and non-animal origin. In some aspects, sources of non-animal origin include yeast or bacteria. In some aspects, the molecular weight of a single hyaluronic acid molecule is typically in the range of 0.1 to 10 mDa, but other molecular weights are possible.

In some aspects, the hydrogel is diluted in a PBS buffer. In some aspects, the hydrogel is diluted in a 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM phosphate buffer. In some aspects, the hydrogel is diluted in about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, or about 20 mM phosphate buffer. In some aspects, the hydrogel is diluted to between 1 mM to 20 mM, 1 mM to 15 mM, 1 mM to 10 mM, 1 mM to 5 mM, 5 mM to 20 mM, 5 mM to 15 mM, 5 mM to 10 mM, 10 mM to 20 mM, 10 mM to 15 mM, or 15 mM to 20 mM phosphate buffer. In some aspects, the hydrogel is diluted to between about 1 mM to about 20 mM, about 1 mM to about 15 mM, about 1 mM to about 10 mM, about 1 mM to about 5 mM, about 5 mM to about 20 mM, about 5 mM to about 15 mM, about 5 mM to about 10 mM, about 10 mM to about 20 mM, about 10 mM to about 15 mM, or about 15 mM to about 20 mM phosphate buffer.

In some aspects, the hydrogel is diluted in a solution at a pH of about 6.0, about 6.2, about 6.4, about 6.6, about 6.8, about 7.0, about 7.2, about 7.4, about 7.6, about 7.8, or about 8.0. In some aspects, the hydrogel is diluted in a solution at a pH of 6.0, 6.2, 6.4, 6.6, 6.8, 7.0, 7.2, 7.4, 7.6, 7.8, or 8.0. In some aspects, the hydrogel is diluted in a solution at a pH of between 6.0 to 8.0, between 6.0 to 7.0, between 7.0 and 8.0, between 6 and 7.5, between 7.0 and 7.5, or between 6.5 and 7.5.

In some aspects, the HA concentration of the crosslinking reaction is about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20%. In some aspects, the HA concentration of the crosslinking reaction is 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10% 11%, 12%, 13%, 14%, 15%, 16%, 17%, 18%, 19%, or 20%. In some aspects, the HA concentration of the crosslinking reaction is between 1% to 3%, 2% to 5%, 3% to 5%, 4% to 5%, 2% to 3%, 2% to 4%, 3% to 5%, 3% to 4%, 1% to 20%, 1% to 15%, 1% to 10%, 1% to 5%, 1% to 2%, 2% to 5%, 2% to 10%, 2% to 15%, 2% to 20%, 5% to 20%, 5% to 15%, 5% to 10%, 10% to 20%, 10% to 15%, or 15% to 20%.

In some aspects, the HA concentration of the crosslinking reaction is not greater than 10%. In some aspects, the HA concentration of the crosslinking reaction is not greater than 9%. In some aspects, the HA concentration of the crosslinking reaction is not greater than 8%. In some aspects, the HA concentration of the crosslinking reaction is not greater than 7%. In some aspects, the HA concentration of the crosslinking reaction is not greater than 6%. In some aspects, the HA concentration of the crosslinking reaction is not greater than 5%. In some aspects, the HA concentration of the crosslinking reaction is not greater than 4%. In some aspects, the HA concentration of the crosslinking reaction is not greater than 3%.

In some aspects, the mol-% of crosslinker in the crosslinking reaction is 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, or 2%. In some aspects, the mol-% of the crosslinker in the crosslinking reaction is about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2%. In some aspects, the mol-% of the crosslinker in the crosslinking reaction is 0.1% to 2%, 0.1% to 1.5%, 0.1% to 1%, 0.1% to 0.5%, 0.2% to 1%, 0.2% to 0.5%, 0.2% to 0.3%, 0.5% to 1%, 0.5% to 1.5%, 0.5% to 2%, 1% to 2%, or 1% to 1.5%.

In some aspects, the GAG has a molecular weight of above 100 kDa, 200 kDa, 300 kDa, 400 kDa, 500 kDa, 600 kDa, 700 kDa, 800 kDa, 900 kDa, 1000 kDa, 1100 kDa, 1200 kDa, 1300 kDa, 1400 kDa, 1500 kDa, 1600 kDa, 1700 kDa, 1800 kDa, 1900 kDa, 2000 kDa, 2500 kDa, 3000 kDa, 3500 kDa, 4000 kDa, 4500 kDa, 5000 kDa, 5500 kDa, 6000 kDa, 6500 kDa, 7000 kDa, 7500 kDa, 8000 kDa, 8500 kDa, 9000 kDa, 9500 kDa, or 10000 kDa.

In some aspects, the GAG has a molecular weight of above about 100 kDa, about 200 kDa, about 300 kDa, about 400 kDa, about 500 kDa, about 600 kDa, about 700 kDa, about 800 kDa, about 900 kDa, about 1000 kDa, about 1100 kDa, about 1200 kDa, about 1300 kDa, about 1400 kDa, about 1500 kDa, about 1600 kDa, about 1700 kDa, about 1800 kDa, about 1900 kDa, about 2000 kDa, about 2500 kDa, about 3000 kDa, about 3500 kDa, about 4000 kDa, about 4500 kDa, about 5000 kDa, about 5500 kDa, about 6000 kDa, about 6500 kDa, about 7000 kDa, about 7500 kDa, about 8000 kDa, about 8500 kDa, about 9000 kDa, about 9500 kDa, or about 10000 kDa.

In some aspects, the thermostability (NormGelC or GelC) of the hydrogel is at least 80% after 24 hours. In some aspects, the thermostability (NormGelC or GelC) of the hydrogel is at least 80% after 48 hours. In some aspects, the thermostability (NormGelC or GelC) of the hydrogel is at least 80% after 24 hours at about 90° C. In some aspects, the thermostability (NormGelC or GelC) of the hydrogel is at least 80% after 24 or 48 hours at a temperature of at least 70° C. In some aspects, the thermostability (NormGelC or GelC) of the hydrogel is at least 80% after 24 or 48 hours at a temperature of at least 90° C.

In some aspects, the thermostability (NormGelC or GelC) of the hydrogel is at least 70%, 75%, 80%, 85%, 90%, or 95% after 24 hours or 48 hours. In some aspects, the thermostability (NormGelC or GelC) of the hydrogel is at least 70%, 75%, 80%, 85%, 90%, or 95% after 24 hours or 48 hours at a temperature of at least 70° C. or at least 90° C.

In some aspects, the thermostability (NormGelC or GelC) of the hydrogel is at least about 70%, about 75%, about 80%, about 85%, about 90%, or about 95% after 24 hours or 48 hours. In some aspects, the thermostability (NormGelC or GelC) of the hydrogel is at least 70%, 75%, 80%, 85%, 90%, or 95% after 24 hours or 48 hours at a temperature of at least 70° C. or at least 90° C.

In some aspects, the thermostability (NormGelC or GelC) of the hydrogel decreases by less than 5%, 10%, 15%, 20%, 25%, or 30% after 24 hours or 48 hours at a temperature of at least 70° C. or at least 90° C. In some aspects, the thermostability (NormGelC or GelC) of the hydrogel decreases by less than about 5%, about 10%, about 15%, about 20%, about 25%, or 30% after 24 hours or 48 hours at a temperature of at least 70° C. or at least 90° C.

In some aspects, the temperature at which the thermostability is determined is at least 70° C., 72° C., 74° C., 76° C., 78° C., 80° C., 82° C., 84° C., 86° C., 88° C., 90° C., 92° C., 94° C., 96° C., 98° C., 100° C., 102° C., 104° C., 106° C., 108° C., or 110° C. In some aspects, the temperature at which the thermostability is determined is at least about 70° C., about 72° C., about 74° C., about 76° C., about 78° C., about 80° C., about 82° C., about 84° C., about 86° C., about 88° C., about 90° C., about 92° C., about 94° C., about 96° C., about 98° C., about 100° C., about 102° C., about 104° C., about 106° C., about 108° C., or about 110° C.

In some aspects, the period of time post-manufacture at which the thermostability is determined is at least 24 hours, 26 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, 72 hours, 74 hours, 76 hours, 78 hours, or 80 hours. In some aspects, the period of time post-manufacture at which the thermostability is determined is at least about 24 hours, about 26 hours, about 28 hours, about 30 hours, about 32 hours, about 34 hours, about 36 hours, about 38 hours, about 40 hours, about 42 hours, about 44 hours, about 46 hours, about 48 hours, about 50 hours, about 52 hours, about 54 hours, about 56 hours, about 58 hours, about 60 hours, about 62 hours, about 64 hours, about 66 hours, about 68 hours, about 70 hours, about 72 hours, about 74 hours, about 76 hours, about 78 hours, or about 80 hours.

In some aspects the composition is bioresorbable. In some aspects, the hydrogel is bioresorbable. In some aspects, the composition is bioresorbed within a period of about 1 year to about 3 years. In some aspects, the composition is bioresorbed within a period of 1 year to 3 years. In some aspects, the hydrogel is bioresorbed within a period of about 1 year to about 3 years. In some aspects, the hydrogel is bioresorbed within a period of 1 year to 3 years.

In some aspects, the composition further comprises a local anesthetic. In some aspects, the composition comprises at least one local anesthetic. In some aspects the local anesthetic is an amide-type local anesthetic. In some aspects, the local anesthetic is an ester-type local anesthetic.

In some aspects, the local anesthetic is selected from the group consisting of: bupivacaine, butanilicaine, carticaine, cinchocaine (dibucaine), clibucaine, ethyl parapiperidinoacetylaminobenzoate, etidocaine, lignocaine (lidocaine), mepivacaine, oxethazaine, prilocaine, ropivacaine, tolycaine, trimecaine, vadocaine, articaine, levobupivacaine, amylocaine, cocaine, propanocaine, clormecaine, cyclomethycaine, proxymetacaine, amethocaine (tetracaine), benzocaine, butacaine, butoxycaine, butyl aminobenzoate, chloroprocaine, dimethocaine (larocaine), oxybuprocaine, piperocaine, parethoxycaine, procaine (novocaine), propoxycaine, and tricaine; or a combination thereof.

In some aspects, the concentration of local anesthetic in the composition is between 1 to 5 mg/mL. In some aspects, the concentration of local anesthetic in the composition is between about 1 to about 5 mg/mL. In some aspects, the concentration of local anesthetic in the composition is between 2 to 4 mg/mL. In some aspects, the concentration of local anesthetic in the composition is between about 2 to about 4 mg/mL. In some aspects, the concentration of local anesthetic in the composition is 0.5 mg/mL, 1 mg/mL, 1.5 mg/mL, 2 mg/mL, 2.5 mg/mL, 3 mg/mL, 3.5 mg/mL, 4 mg/mL, 4.5 mg/mL, or 5 mg/mL. In some aspects, the concentration of local anesthetic in the composition is about 0.5 mg/mL, about 1 mg/mL, about 1.5 mg/mL, about 2 mg/mL, about 2.5 mg/mL, about 3 mg/mL, about 3.5 mg/mL, about 4 mg/mL, about 4.5 mg/mL, or about 5 mg/mL.

In some aspects, the composition comprises one or more pharmaceutical agents selected from one or more of a local anesthetic, an analgesic, an anti-inflammatory drug, a hormone, a steroid, a blood clotting agent, or an antibiotic, among others.

In some aspects, the composition comprises one or more living cells. In some aspects, the composition comprises one or more living mammalian cells. In some aspects, the composition comprises one or more living human cells. In some aspects, the composition comprises one or more living cells that have been genetically modified. In some aspects, the composition comprises one or more living genetically modified cells comprising one or more heterologous nucleic acid sequences.

In some aspects, the composition is injectable. In some aspects, the injectable composition is an injectable implant. In some aspects, the disclosure is drawn to an injectable implant comprising any one of the compositions disclosed herein. In some aspects, the injectable implant is for subdermal, intradermal, subcutaneous, intramuscular, submuscular, intragingival injection.

In some aspects, the disclosure is drawn to a pre-filled syringe comprising any one of the compositions disclosed herein. In some aspects, the disclosure is drawn to a pre-filled vial comprising any one of the compositions disclosed herein.

In some aspects, a kit comprises a pre-filled syringe comprising any one of the compositions disclosed herein. In some aspects, a kit comprises a pre-filled vial comprising any one of the compositions disclosed herein, a syringe, and one or more hypodermic needles. In some cases the kit comprises an antimicrobial composition for administering to the site of injection.

In some aspects, kits for use in practicing the methods described herein are contemplated. In some aspects, kits comprise all solutions, buffers, compounds, vessels, and/or instructions sufficient for performing the methods described herein.

In some aspects, the composition further comprises sodium chloride. In some aspects, the composition exhibits a sodium chloride concentration of 0.9% w/v. In some aspects, the composition further comprises a phosphate buffer. In some aspects, the composition further comprises a pharmaceutically acceptable carrier. In some aspects the composition further comprises sodium chloride, a phosphate buffer, and a pharmaceutically acceptable carrier.

In some aspects, the composition comprises one or more density enhancing agents. In some aspects, the density enhancing agents may be selected from sorbitol, mannitol, and fructose.

In some aspects, the composition comprises a buffering agent. A buffering agent is a chemical compound that is or compounds that are added to a solution to allow that solution to resist changes in pH as a result of either dilution or small additions of acids or bases. Effective buffer systems employ solutions which contain large and approximately equal concentrations of a conjugate acid-base pair (or buffering agents). A buffering agent employed herein may be any such chemical compound(s) which is pharmaceutically acceptable, including but not limited to salts (conjugates acids and/or bases) of phosphates and citrates. In some aspects, the buffering agent comprises phosphate buffered saline (PBS) or an alternative phosphate buffer.

In some aspects, the composition is aseptic. In some aspects, the composition is sterile. In some aspects, the composition is sterilized via filtration sterilization, heat sterilization, or irradiation sterilization. In some aspects, components of the composition are sterilized prior to mixing or forming the whole composition, thus resulting in a composition that comprises two or more components that were sterilized prior to forming the composition.

In some aspects, the GAG does not have a molecular weight of less than 1.5 MDa. In some aspects, the GAG does not have a molecular weight of less than 1.4 MDa. In some aspects, the GAG does not have a molecular weight of less than 1.3 MDa. In some aspects, the GAG does not have a molecular weight of less than 1.2 MDa. In some aspects, the GAG does not have a molecular weight of less than 1.1 MDa. In some aspects, the GAG does not have a molecular weight of less than 1.0 MDa. In some aspects, the GAG does not have a molecular weight of less than 0.9 MDa. In some aspects, the GAG does not have a molecular weight of less than 0.8 MDa. In some aspects, the GAG does not have a molecular weight of less than 0.7 MDa.

In some aspects, the hydrogel is not subjected to a post-crosslinking degradation of the glycosaminoglycan. In some aspects, the hydrogel is subject to ambient degradation post-crosslinking; however, the hydrogel does not exhibit a Cₘᵢₙ value below that of C_{final}/2. In some aspects, the hydrogel exhibits a Cₘᵢₙ value greater than C_{final}/2 of the hydrogel.

Other aspects and preferred embodiments of the present invention will be evident from the following detailed disclosure of the invention and the appended claims.

### III. Methods of Using the Hydrogels

In some aspects, the present disclosure comprises methods of performing reparative or esthetic dermatologic treatment. In some aspects, the reparative or esthetic dermatologic treatment comprises injecting a subject with a composition disclosed herein. In some aspects, the injection is a subdermal, intradermal, subcutaneous, intramuscular, submuscular, or intragingival injection.

In some aspects, methods of the present disclosure are drawn to intragingival injection to fill the gums as a result of receding gums. In some aspects, methods are drawn to injection of the composition in one or more tissues of the oral cavity.

In some aspects, the injection is for dermal filling, body contouring, facial contouring, and gingival filling.

In some aspects, the injection of a composition disclosed herein is for dermal filling. In some aspects, methods of dermal filling include injection of the composition to fill skin cracks. In some aspects, methods of dermal filling include injection of the composition to fill fine lines in the face, neck, hands, feet, knees, and elbows. In some aspects, methods of dermal filling include injection of the composition to fill fine wrinkles in the face, neck, hands, feet, knees, and elbows. In some aspects, methods of dermal filling include injection of the composition to fill fine lines in the face, neck, hands, feet, knees, and elbows.

In some aspects, methods of dermal filling include injection of the composition to fill scars. In some aspects, methods of dermal filling include injection of the composition to fill depressed scars. In some aspects, methods of dermal filling include injection of the composition to fill hypertrophic scars. In some aspects, methods of dermal filling include injection of the composition to fill keloid scars.

In some aspects, methods of dermal filling include injection of the composition to restore and/or correct for signs of facial fat loss (lipoatrophy) in people with human immunodeficiency virus (HIV).

In some aspects, methods of dermal filling include injection of the composition to the backs of hands or the top of feet.

In some aspects, methods of dermal filling include injection of the composition to strengthen weakened vocal cords.

In some aspects, methods of dermal filling include injection of the composition to restore lost volume to a portion of the body as a result of age, illness, or injury.

In some aspects, methods of facial contouring include injection of the composition to the face to modify the facial contour. In some aspects, methods of facial contouring include injection of the composition to the lips to augment the size and/or shape of the lips.

In some aspects, methods of facial contouring include injection of the composition to the face to increase facial symmetry. In some aspects, methods of facial contouring include injection of the composition to change the shape of the face to an oval shape, round shape, square shape, triangle shape, inverted triangle shape, rectangular shape, or oblong shape. In some aspects, methods of facial contouring include injection of the composition to increase the total width of the face. In some aspects, methods of facial contouring include injection of the composition to increase the total length of the face.

In some aspects, methods of facial contouring include injection of the composition to the face to increase the forehead and/or checkbone width. In some aspects, methods of facial contouring include injection of the composition to the face to increase the length of the jawline.

In some aspects, methods of facial contouring include injection of the composition to the face to change the size and/or shape of the chin. In some aspects, methods of facial contouring include injection of the composition to the face to change the size and/or shape of the forehead. In some aspects, methods of facial contouring include injection of the composition to the face to change the size and/or shape of the cheeks. In some aspects, methods of facial contouring include injection of the composition to the face to change the size and/or shape of the brow.

In some aspects, methods of facial contouring include injection of the composition to the face to modify the appearance associated with retrognathia. In some aspects, methods of facial contouring include injection of the composition to the face to modify the appearance associated with prognathism.

In some aspects, methods of body contouring include injection of the composition to the body to modify the size and shape of various aspects of the body. In some aspects, methods of body contouring include injection of the composition to the body to modify the size and shape of aspects of the body to increase symmetry.

In some aspects, methods of body contouring include injection of the composition to the body to modify the size and shape of the breasts, buttocks, sacrum, groin, hips, abdomen, thorax, feet, legs, knees, popliteus, thighs, arms, hands, elbows, and/or antecubitis,

In some aspects, methods of body contouring include injection of the composition to the body to fill a concave deformity. In some aspects, the concave deformity is a result of age, illness, injury, or predisposition. In some aspects, methods of body contouring include injection of the composition to the body to decrease the appearance of cellulite.

### EXAMPLES

It has been observed that the sensitivity to change in temperature (activation energy, Ea, Arrhenius slope) is higher for the degradation (depolymerization) of HA than for the reaction rate of BDDE. This led to the idea that by changing the crosslinking temperature, while adjusting reaction time to allow the same level of crosslinking, the degradation level of HA during crosslinking can be adjusted. The expected result is that crosslinking at lower temperatures would result in a gel with longer HA chains, and conversely, that crosslinking at higher temperatures would result in a gel with shorter HA chains.

To demonstrate this, three crosslinking experiments were performed at temperatures 5° C., 23° C. and 50° C., where the reaction time was adjusted to give the same level of crosslinking. The crosslinking conditions, apart from time and temperature, were set according to NASHA.

The length of the HA chains in the crosslinked gels were estimated by analyzing the Mw of HA solutions from reference “crosslinking” experiments with no crosslinker. The properties of the final crosslinked gel were studied by testing the parameters rheology, swelling factor (SwF) and gel content (GelC). The level of crosslinking was studied by measuring the degree of modification (MoD).

### Example 1

### Sample Preparation

The HA was weighed into a PTFE container. NaOH was dissolved in H2O and BDDE was added. The NaOH/BDDE solution was added, and the container was shaken in a paint shaker for 3.5 minutes. The concentrations are tabled in Table 1. The container was placed in an incubator or in a water bath for the crosslinking process, with temperature and time as given in Table 1. Subsequently the highly concentrated gel “puck” was divided into particles (PSR, particle size reduction) by pressing it through a steel mesh with a 1 mm grid size, using the “puckpress”.

The gel particles were allowed to swell to a concentration of about 20 mg/g in 0.9% NaCl and neutral pH for about 20 hours at 70° C. in order to terminate the crosslinking process and let any still active BDDE turn into non-reactive BDPE, or to BDDE derivatives that are not linked to HA. The gel was passed through a steel mesh with a grid size of 315 μm, precipitated in ethanol to remove residual crosslinker and derivatives, and dried. The dried gel powder was swollen in 0.9% NaCl to about 25 mg/ml in order to avoid over-saturation of the gel. The swelling media contained 7 mM of phosphate buffer.

### HA Solutions

In order to estimate the degradation rates of HA chains during crosslinking and final HA chain length in the crosslinked gel, HA solutions were prepared and treated similarly to the gel samples, including reaction time, swelling at 70° C. After reaction time, the solutions were neutralized and diluted to about 1% HA in 0.9% NaCl with 1 mM phosphate buffer. The solutions were not autoclaved, in order to make estimations of degradation rates during crosslinking more exact. For simplicity, the Mw of the HA solution without autoclaving were used also as an approximate estimate of HA chain length in the final gel.

TABLE 1
Gel samples and crosslinking conditions

| | | | HA | NaOH | NaOH |
|---|---|---|---|---|---|
| | | BDDE | Crosslink³ | Crosslink² | Solution¹ |
| Temp (° C.) | Time (h) | NASHA = 1 | (w/w %) | (w/w %) | (w/w %) |
| 50 | 2 | 1 | 25 | 0.75 | 1.0 |
| 23 | 24 | 1 | 25 | 0.75 | 1.0 |
| 5 | 196 | 1 | 25 | 0.75 | 1.0 |

¹NaOH concentration of the solution added
²NaOH concentration during crosslinking when mixed with HA
³HA concentration during crosslinking

TABLE 2
HA solutions

| | | | HA | NaOH | NaOH |
|---|---|---|---|---|---|
| | | BDDE | Crosslink³ | Crosslink² | Solution¹ |
| Temp (° C.) | Time (h) | NASHA = 1 | (w/w %) | (w/w %) | (w/w %) |
| 50 | 2 | 0 | 25 | 0.75 | 1.0 |
| 23 | 24 | 0 | 25 | 0.75 | 1.0 |
| 5 | 196 | 0 | 25 | 0.75 | 1.0 |

¹NaOH concentration of the solution added
²NaOH concentration in the “puck” when mixed with HA
³HA concentration during crosslinking

### Example 2

### Analytical Test Methods

The compositions were evaluated for the following parameters: MW (SEC-UV), rheometry, SwF, MOD, CrR, GelC, HA concentration, and CrD.

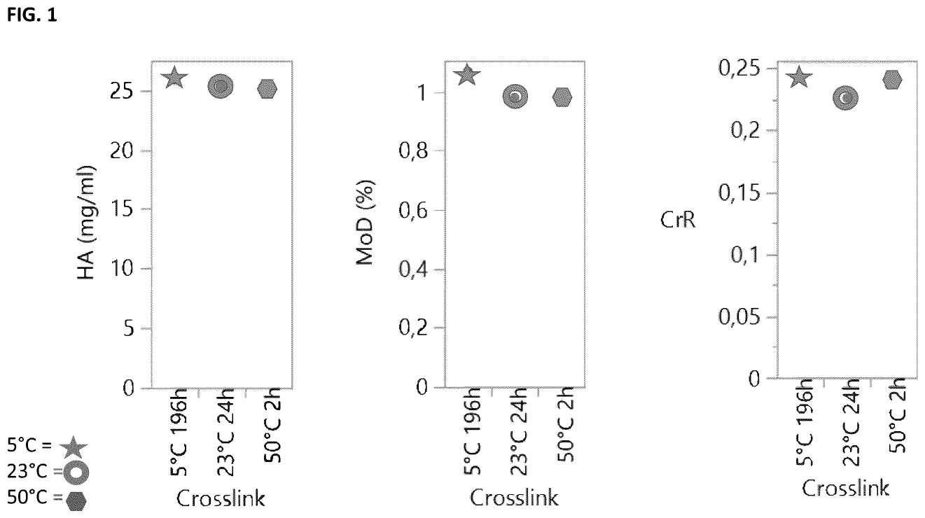

All three experiments resulted in similar levels of MOD, which means that the reaction times were accurate enough to yield similar levels of completion of crosslinking for all experiments. Crosslinking efficiency ratio (CrR), and the concentration of HA was also similar between preparations (FIG. 1).

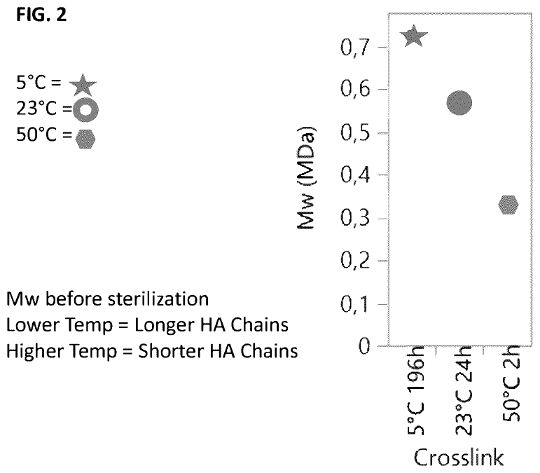

The HA chain length of crosslinked product, as given by Mw from reference experiments, decreased noticeably when crosslinking at increasing temperatures (FIG. 2). Again, it should be noted that the solutions were not autoclaved. Since the degradation rates during crosslinking was of main interest, the Mw of the HA solution without autoclaving were used as an approximate estimate of HA chain length in the final gel.

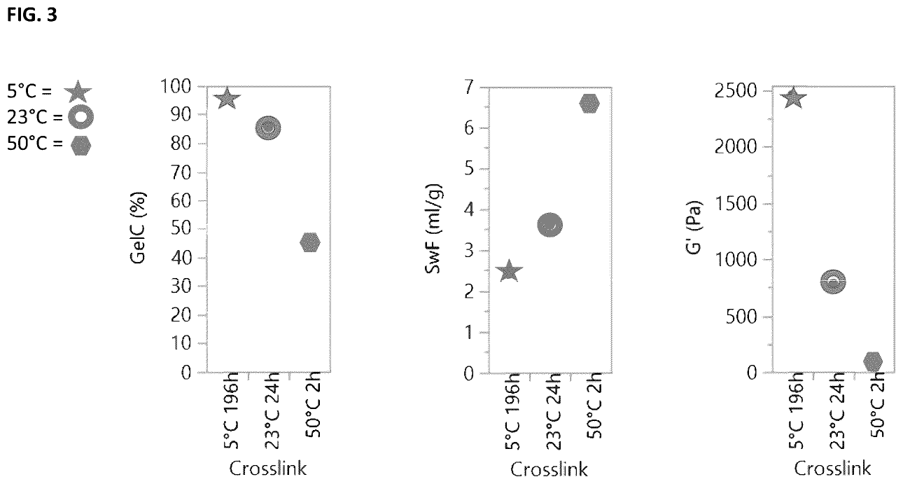

The decrease in HA chain length had an obvious impact on the gel strength characteristics, as shown by decreasing G′ and GelC, as well as increasing SwF (FIG. 3). The decrease in G′ was especially large, probably a combined effect of a weaker gel, as given by the increase in SwF, as well as the decrease in GelC, since it is known that an increasing amount of free HA can lower the G′ to a great extent.

It can be noted that with a difference in HA chain length a factor of two between the 5 and the 50° C. experiments, the difference in gel properties were considerable, with G′ differing by a factor of 50. This demonstrates that differences in the final HA gel chain length can have a great impact on gel properties.

### Example 3

Prediction of Reaction Rates for BDDE Reacting with HA

The required BDDE reaction times were calculated based on the Ea value 72 KJ/mol, which was obtained for BDDE reacting with NaOH (actually NaOD for NMR analysis) without HA present. Based on this Ea value, the reaction time corresponding to 24 h at 23° C. was 159 h for 5° C., about 6.6 days, and 2.1 h for 50° C.

In practice, the 5° C. reaction was allowed to go for 9 days, in order to obtain at least the same level of crosslinking as for NASHA, considering the uncertainty of the Ea value and its great impact on the calculation. The 50° C. reaction was allowed to go for 2.1 h, close to the 2 h used for the OBT process. As shown by the similarity in obtained MOD values, the predictions appeared to be reasonably accurate.

The predicted required reaction times for BDDE, compared to 24 h at 23° C., based on Ea of 72 KJ/mol. The 5° C. reaction was predicted to require 159 hours. The 23° C. reaction was predicted to require 24 hours. The 50° C. reaction was predicted to require 2.1 hours.

### Example 4

### Prediction of HA Degradation Rates

For prediction of HA degradation rates, an Ea value of 100 KJ/mol was used, obtained from degradation experiments on reference crosslinking experiments at different combinations of high concentrations of HA and NaOH, without BDDE. Similar to the calculations made for BDDE, the corresponding reaction time to reach similar HA degradation levels between temperatures were calculated (Table 3). The higher Ea value used for this calculation results in greater differences between temperatures, requiring 333 h to degrade HA at 5° C. to the same level as when degrading for 24 h at 23° C. At 50° C., the required time is only 0.8 h. Since the reaction times were chosen to achieve equal levels of crosslinking, less degradation of HA was expected for the 5° C. experiment, and more degradation for the 50° C. experiment.

TABLE 3
Corresponding degradation times for HA, compared to
24 h at 23° C., and based on Ea of 100 kJ/mol.

| Temp (° C.) | Time (h) | Rate Ratio to 23° C. |
|---|---|---|
| 5 | 333 | 0.0719 |
| 23 | 24 | 1 |
| 50 | 0.8 | 29.8 |

### Example 5

### Resulting HA Degradation Rates and Arrhenius Slope

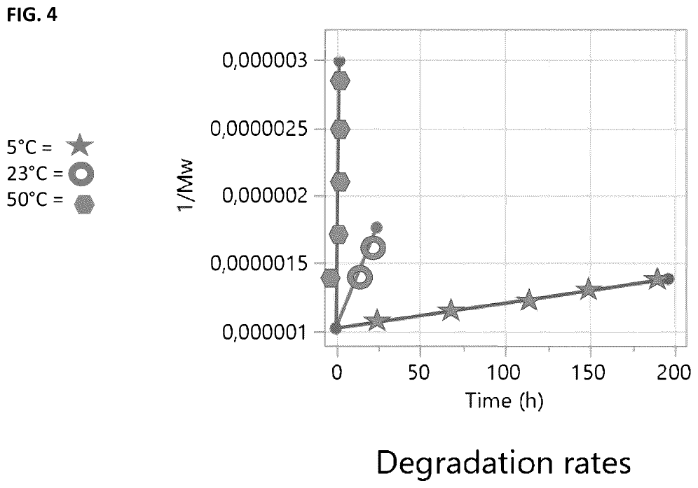

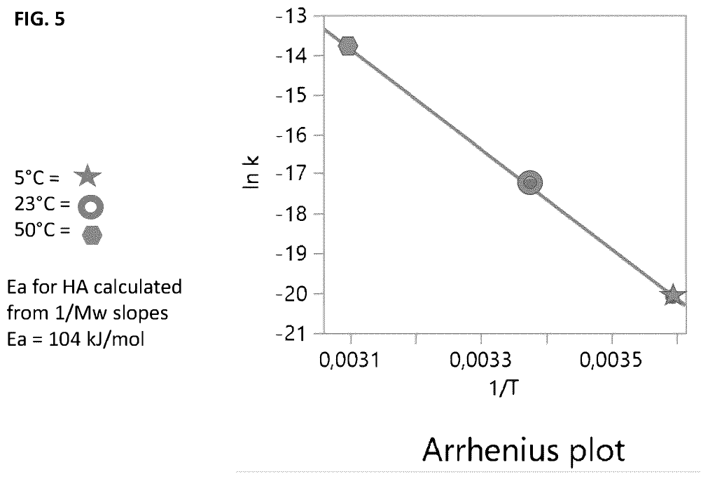

A linear correlation of 1/Mw against time shows the magnitude of difference in degradation rate between temperatures (FIG. 4). The slopes 1/Mw describe the degradation rates (Table 4). The rates plotted vs temperature according to Arrhenius gave an excellent linear fit (r2=1.00), yielding an Ea value of 104 KJ/mol, close to the 100 KJ/mol used in setting experimental conditions (FIG. 5).

TABLE 4
Degradation Rates k (slope of 1/Mw)

| Temp (° C.) | Rate k (mol/(g*h) | Sloe Ratio to 23° C. |
|---|---|---|
| 5 | 1.870E−09 | 0.0602 |
| 23 | 3.103E−09 | 1 |
| 50 | 9.888E−09 | 31.9 |

### Example 6

### Comparison of Mw Over Time

In spite of the actual degradation rate being constant over time, Mw vs time is strongly non-linear. The curvature is caused by the fact that for each chain that is cut in two, two more chains has to be cut in two, in order to decrease Mw a factor of 2 again. With time, an immense number of chains have to break in order to decrease the Mw further. The result is that the degradation viewed as Mw versus time appears to slow down over time.

In degradation experiments, it is often of interest to compare Mw between experiments. The fact that 1/Mw vs time is linear, while Mw vs time is not, makes it less intuitive to envision the effect of degradation over time, even when knowing the degradation rates. An attempt to visualize the effect is given below.

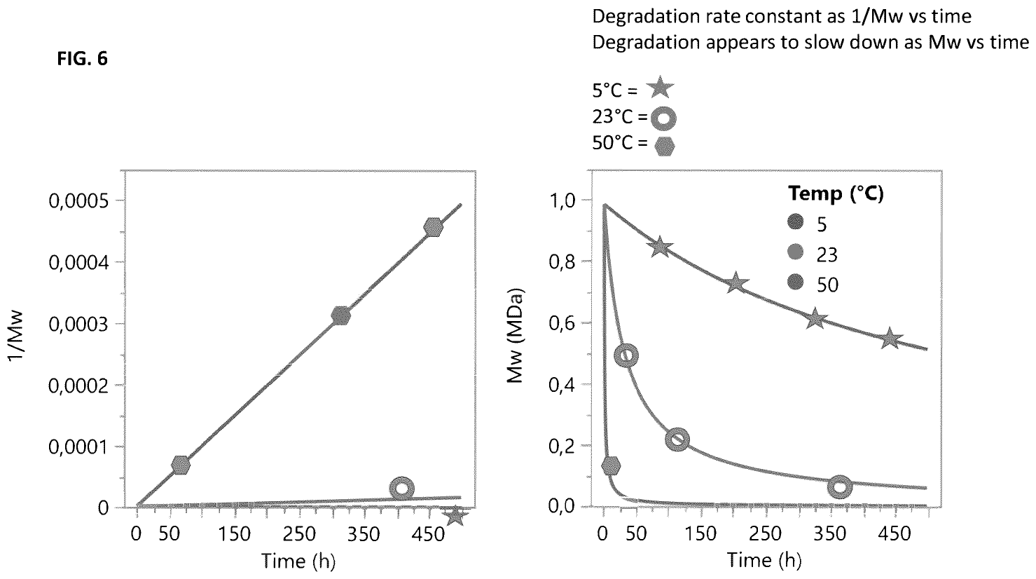

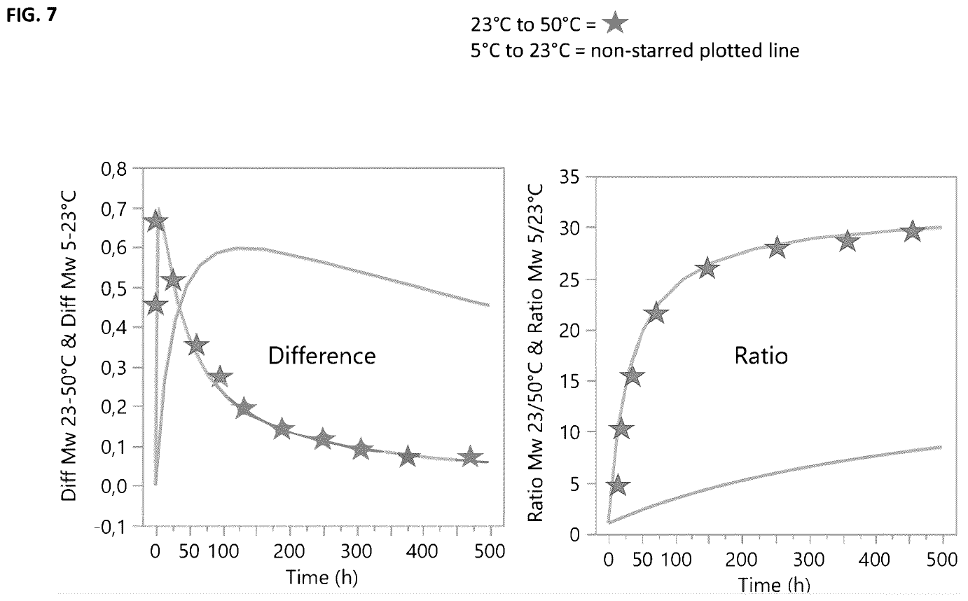

Degradation shown as Mw versus time for this experiment, extrapolated to 500 h, shows the strong curvature of Mw over time, compared to the linear 1/Mw (FIG. 6). The numerical difference in Mw between the 5 and 23° C., as well as the 23 and 50° C. experiments, was calculated for each time point (FIG. 7, left). It can be seen that the difference starts at zero, as no degradation has yet occurred, increasing over time as the higher temperature causes Mw to decrease faster. The peak difference occurs at 130 h, comparing the lower temperatures, and about 6 h, comparing the higher temperatures.

The ratio of Mw between temperatures was also calculated (FIG. 7, right). From this perspective, there is no maximum. The ratio of Mw between temperatures keeps increasing indefinitely, the higher temperatures causing a larger ratio.

### Example 7

### Calculation of Relative Reaction Rates

The expected increase in reaction rate from a certain increase in temperature can be calculated by applying a known value of Ea and the high and low temperatures into the Arrhenius equation. The ratio of the high to the low reaction rate (Eq. 1) is calculated by replacing k with the right hand part of Eq. 2, resulting in Eq. 3. The pre-exponential or frequency factor (A) can be left out, since it will cancel in the ratio equation.

k=the rate constant (i.e., the reaction/degradation rate)
Ea=the activation energy in J/mol
R=the gas constant (8.3145 J*mol⁻¹*K⁻¹)
A=the pre-exponential or frequency factor
T=the temperature in K
T_{high}=the high temperature
T_{low}=the low temperature

$$Ratio = \frac{{k}_{high}}{{k}_{low}} \qquad Eq . 1$$

$$k = {Ae}^{- \frac{Ea}{R * T}} \qquad Eq . 2$$

$$Ratio = \frac{{e}^{- \frac{Ea}{R * {T}_{high}}}}{{e}^{- \frac{Ea *}{R * {T}_{low}}}} \qquad Eq . 3$$

Crosslinking using “NASHA” conditions at temperatures 5° C., 23° C., and 50° C. showed that 5° C. resulted in a very firm gel with long HA chains, while crosslinking at 50° C. resulted in a very soft gel with short HA chains. The 23° C. crosslinking resulted in typical NASHA gel properties.

Since all three gels were equal in level of crosslinking and HA concentration, the difference in gel properties were considered mainly due to HA chain length. The final HA gel chain length for the 5° C. crosslinked gel was about two times that of the 50° C. crosslinked gel. The resulting G′ differed by a factor of 50, demonstrating that differences in chain length of the final HA gel can have a great impact on gel properties.

The degradation rates differed by 500 times between the 5 and the 50° C. experiments, yielding an Arrhenius slope of 104 KJ/mol, close to the previously determined 100 KJ/mol. The difficulty to envision the effect of degradation over time, due to the non-linearity of Mw over time, was discussed and demonstrated.

The data suggests that the molecular weight and the resulting gel properties can be adjusted by temperature during crosslinking.

The 5° C. crosslinking temperature resulted in a very firm gel with long HA chains.

The 50° C. crosslinking temperature resulted in a very soft get with short HA chains.

The final Mw_{5° C.} is about 2 times Mw_{50° C.}.

The final G′_{5° C.} is about 50 times G′_{50° C.}.

Ea=104 KJ/mol for HA degradation at high OH concentration.

Comparing Mw and degradation rates between temperatures is not intuitive, must calculate.

The methods illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof. It is recognized that various modifications are possible within the scope of the disclosure claimed. Thus, it should be understood that although the present disclosure has been specifically disclosed by preferred embodiments and optional features, modification and variation of the disclosure embodied therein herein disclosed may be resorted to by those skilled in the art, and that such modifications and variations are considered to be within the scope of this disclosure.

The disclosure has been described broadly and generically herein. Each of the narrower species and subgeneric groupings falling within the generic disclosure also form part of the methods. This includes the generic description of the methods with a proviso or negative limitation removing any subject matter from the genus, regardless of whether or not the excised material is specifically recited herein. The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

One skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

In addition, where features or aspects of the disclosure are described in terms of Markush groups, those skilled in the art will recognize that the disclosure is also thereby described in terms of any individual member or subgroup of members of the Markush group.

All references, articles, publications, patents, patent publications, and patent applications cited herein are incorporated by reference in their entireties for all purposes.

However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed:

1. A process for preparing a product comprising crosslinked glycosaminoglycan (GAG) molecules, the process comprising:
(a) crosslinking GAG molecules with a diepoxide crosslinking agent at a pH of at least 8.5 at a temperature of less than 20° C., and
(b) swelling the crosslinked GAG molecules in an aqueous solution.

2. The process according to claim 1, wherein the diepoxide crosslinking agent is 1,4-butanediol diglycidyl ether.

3. The process according to claim 1, wherein the crosslinking occurs for at least 2 hours.

4. The process according to claim 1, wherein the crosslinking occurs for at least 6 days.

5. The process according to claim 1, wherein gel particles are formed from the crosslinked GAG molecules prior to (b).

6. The process according to claim 1, wherein gel particles are formed from the crosslinked GAG molecules after (b).

7. The process according to claim 1, wherein the crosslinking is performed at a pH of at least 9.

8. The process according to claim 1, wherein sodium hydroxide is present during the crosslinking at a concentration of about 0.5 w/w % to about 10 w/w %.

9. The process according to claim 1, comprising adjusting the pH to a neutral pH after crosslinking.

10. The process according to claim 1, wherein the GAG molecules are hyaluronic acid (HA) molecules.

11. The process according to claim 1, wherein the GAG molecules are present at about 5 w/w % to about 50 w/w % during crosslinking.

12. The process according to claim 1, wherein the aqueous solution comprises a dissolved salt.

13. The process according to claim 1, wherein the aqueous solution comprises a buffer.

14. The process according to 13, wherein the buffer comprises a phosphate buffer.

15. The process according to claim 1, wherein the degradation rate of HA chains during cross-linking is about 500 times lesser than a degradation rate of HA chains during crosslinking at a pH of at least 8.5 at a temperature of about 50° C.

16. The process according to claim 1, wherein the crosslinking is performed at about 5° C.

17. A process for preparing a product comprising crosslinked hyaluronic acid (HA) molecules, the process comprising:
(a) crosslinking HA molecules with 4-butanediol diglycidyl ether crosslinking agent under alkaline conditions at a temperature of about 5° C. or about 50° C. for between about 2 to about 200 hours,
(b) dividing the crosslinked HA molecules into particles of about 1 mm,
(c) swelling the crosslinked HA from (b) in about 0.9% NaCl until the particles reach a concentration of about 20 mg/g, and
(d) collecting and sterilizing the crosslinked HA from (c).

18. The process of claim 17, wherein the crosslinking is performed at about 5° C.

19. The process of claim 18, wherein the crosslinking is performed for at least about 9 days.

20. A process for preparing a product comprising crosslinked glycosaminoglycan (GAG) molecules, the process comprising:
(a) crosslinking GAG molecules with a diepoxide crosslinking agent at a pH of at least 8.5 at a temperature of greater than 45° C. for at least 2 hours, and
(b) swelling the crosslinked GAG molecules in an aqueous solution.
